(19) United States
(12) Patent Application Publication
(10) Pub. No.: US 2005/0069928 A1
(43) Pub. Date: Mar. 31, 2005

(54) Methods for synthesis of defined polynucleotides

(75) Inventors: Jeffrey S. Nelson, Woodinville, WA (US); John T. Mulligan, Seattle, WA (US); John C. Tabone, Bothell, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Blue Heron Biotechnology, Inc., Bothell, WA (US)

(21) Appl. No.: 10/911,239

(22) Filed: Aug. 3, 2004

Related U.S. Application Data
(60) Provisional application No. 60/492,694, filed on Aug. 4, 2003.

(51) Int. Cl. C12Q 1/68; C07H 21/04; C12P 19/34; A61K 48/00

(52) U.S. Cl. 435/6; 514/44; 435/91.2; 536/25.3

(57) ABSTRACT

Disclosed is a significantly improved synthetic method of producing a set of mutagenized progeny polynucleotides which contain at least one substituted codon encoding for each of the 20 naturally encoded amino acids or any selected subset thereof. This in turn, similarly provides a method for producing from a parental template polypeptide, a set of mutagenized progeny polypeptides in which all 20 naturally encoded amino acids is represented at each original amino acid position or any selected subset thereof. The methods described herein enable the synthesis of defined, complex mixtures of oligonucleotides, in instances where the incorporation of degenerate bases is impractical. These oligonucleotide mixtures are useful for a variety of applications such as recombination methods, site-saturation mutagenesis, or the like.

### CROSS-REFERENCE TO RELATED APPLICATION

This application claims the benefit of U.S. Provisional Patent Application No. 60/492,694, filed Aug. 4, 2003, where this provisional application is incorporated herein by reference in its entirety.

### BACKGROUND OF THE INVENTION

1. Field of the Invention

The invention is in the field of polynucleotide synthesis.

2. Description of the Related Art

Several techniques are available to synthesize complex oligonucleotide mixtures. For example, mixtures of phosphoramidite monomers can be employed to incorporate degenerate bases for applications in which maximal randomness is desired. See, e.g., Zon et al., “Analytical studies of ‘mixed sequence’ oligodeoxyribonucleotides synthesized by competitive coupling of either methyl- or beta-cyanoethyl-N,N-diisopropylamino phosphoramidite reagents, including 2′-deoxyinosine,” Nucleic Acids Res. 13(22): 8181-8196 (1985). Degenerate oligonucleotide primers are commonly used in site-saturation mutagenesis to introduce various mutations into a selected target codon. However, this reportedly renders a mutant population that is biased towards the original nucleotide sequence. Considerable effort has focused on empirically developing rules to balance the ratio of phosphoramidite monomers in the mixture so as to optimize site-saturation mutagenesis, but a generalized and efficient methodology has yet to be realized.

Trinucleotide phosphoramidites representing all 20 amino acid codons have also been described, and can be utilized to generate oligonucleotide/peptide libraries. See, e.g., Virnekas et al., “Trinucleotide phosphoramidites: ideal reagents for the synthesis of mixed oligonucleotides for random mutagenesis,” Nucleic Acids Res. 22(25): 5600-5607 (1994)); Kayushin et al., “A convenient approach to the synthesis of trinucleotide phosphoramidites—synthons for the generation of oligonucleotide/peptide libraries,” Nucleic Acids Res. 24(19): 3748-3755 (1996); and Sondek and Shortle, “A general strategy for random insertion and substitution mutagenesis: Substoichiometric coupling of trinucleotide phosphoramidites,” Proc. Natl. Acad. Sci. USA 89(8): 3581-3585 (1992) This approach is cumbersome however since it requires considerable synthetic effort to generate the required trinucleotide building blocks and has yet to be commercialized, due presumably to high reagent cost and limited success.

The following documents describe compositions and methods related to methods for synthesis of defined polynucleotides. U.S. Pat. No. 6,171,802; PCT International Publication No. WO 01/23401; Murakami et al., “Random insertion and deletion of arbitrary number of bases for codon-based random mutation of DNAs,” Nat. Biotechnol. 20(1): 76-81 (2002); Gayton et al., “Orthogonal combinatorial mutagenesis: a codon-level combinatorial mutagenesis method useful for low multiplicity and amino acid-scanning protocols,” Nucleic Acids Res. 29(3): e9, pages 1-8 (2001); Sawano and Miyawaki, “Directed evolution of green fluorescent protein by a new versatile PCR strategy for site-directed and semi-random mutagenesis,” Nucleic Acids Res. 28(16): e78, pages i-vii (2000); Shin et al., “Effects of saturation mutagenesis of the phage SP6 promoter on transcription activity, presented by activity logos,” Proc. Natl. Acad. Sci. USA 97(8): 3890-3895 (2000); Neuner et al., “Codon-based mutagenesis using dimmer-phosphoramidites,” Nucleic Acids Res. 26(5): 1223-1227 (1998); Airaksinen and Hovi, “Modified base compositions at degenerate positions of a mutagenic oligonucleotide enhance randomness in site-saturation mutagenesis,” Nucleic Acids Res. 26(2): 576-581 (1998); and Zon et al., “Analytical studies of ‘mixed sequence’ oligonucleotides synthesized by competitive coupling of either methyl- or beta-cyanoethyl-N,N-diisopropylamino phosphoramidite reagents, including 2′-deoxyinosine,” Nucleic Acids Res. 13(22): 8181-8196 (1985).

Despite the amount of attention that has been given to these coals, there remains a need in the art for a cost effective, highly efficient, and generalized method for the convergent synthesis of oligonucleotides containing defined regions of heterocyclic base mixtures (dA, dC, dG, and T). The present invention is directed toward fulfilling this need, in that is provides a significantly improved codon-based oligonucleotide synthesis methodology that will prove useful in a number of important biological applications.

### BRIEF SUMMARY OF THE INVENTION

The present invention provides methods of polynucleotide synthesis, including the synthesis of discrete mixtures of polynucleotide in a combinatorial fashion. Existing methods used to generate mixed polynucleotide sets have been previously termed site-saturation mutagenesis or simply saturation mutagenesis, but to date these other methods have by and large utilized complex mixtures containing degenerate base and therefore have limitations, e.g., the undesired introduction of stop codons and the formation of biased polynucleotide mixtures due to the introduction of multiple codons encoding for the same amino acid. Furthermore, other existing methods employed for saturation mutagenesis are incapable of systematically producing a defined subset of polynucleotides containing specific codon insertions.

In one aspect, the present invention provides a method for preparing a polynucleotide, where the method includes:
a) combining a left oligonucleotide (L-ODN), an intermediate oligonucleotide (I-ODN), a right oligonucleotide (R-ODN) and a splint oligonucleotide (S-ODN) to form a mixture, where
   i. a 3′-most region of the L-ODN is functionally complementary to a 3′-most region of the S-ODN;
   ii. a 5′-most region of the R-ODN is functionally complementary to a 5′-most region of the S-ODN;
   iii. the L-ODN and the R-ODN anneal to the S-ODN to provide a gap between the 3′ end of the L-ODN and the 5′ end of the R-ODN;
   iv. the I-ODN anneals to a sequence of nucleotides termed the variable region of the S-ODN and thereby fills the gap; and
b) ligating the I-ODN to both the L-ODN and the R-ODN to form a polynucleotide.

In another aspect, the present invention provides a method for preparing a plurality of polynucleotides, where the method includes:
a) combining a left oligonucleotide (L-ODN), a plurality of intermediate oligonucleotides (I-ODN), a right oligonucleotide (R-ODN) and a splint oligonucleotide (S-ODN) to form a mixture, where
   i. a 3′-most region of the L-ODN is functionally complementary to a 3′-most region of the S-ODN;
   ii. a 5′-most region of the R-ODN is functionally complementary to a 5′-most region of the S-ODN;
   iii. the L-ODN and the R-ODN anneal to the S-ODN to provide a gap between the 3′ end of the L-ODN and the 5′ end of the R-ODN;
   iv. each I-ODN anneals to a sequence of nucleotides termed the variable region of the S-ODN and thereby fills the gap;
   v. members of the plurality of I-ODNs have the same number of nucleotides but differ in their nucleotide sequences; and
b) ligating members of the plurality of I-ODNs to both the L-ODN and the R-ODN to form a plurality of polynucleotides.

In another aspect, the present invention provides a method for preparing a plurality of polynucleotides, where the method includes:
a) combining a left oligonucleotide (L-ODN), a plurality of intermediate oligonucleotides (I-ODNs), a right oligonucleotide (R-ODN) and a plurality of splint oligonucleotide (S-ODNs) to form a mixture, where
   i. a 3′-most region of the L-ODN is functionally complementary to a 3′-most region of the S-ODN;
   ii. a 5′-most region of the R-ODN is functionally complementary to a 5′-most region of the S-ODN;
   iii. the L-ODN and the R-ODN anneal to the S-ODN to provide a gap between the 3′ end of the L-ODN and the 5′ end of the R-ODN;
   iv. each I-ODN anneals to a sequence of nucleotides termed the variable region of the S-ODN and thereby fills the gap;
   v. members of the plurality of I-ODNs have the same number of nucleotides but differ in their nucleotide sequences;
   vi. members of the plurality of S-ODNs have the same number of nucleotides but differ in their nucleotide sequences within the variable region of the S-ODN; and
b) ligating members of the plurality of I-ODNs to both the L-ODN and the R-ODN to form a plurality of polynucleotides.

In another aspect, the present invention provides a composition that includes a left oligonucleotide (L-ODN), an intermediate oligonucleotide (I-ODN), a right oligonucleotide (R-ODN) and a splint oligonucleotide (S-ODN), wherein
i. a 3′-most region of the L-ODN is functionally complementary to a 3′-most region of the S-ODN;
ii. a 5′-most region of the R-ODN is functionally complementary to a 5′-most region of the S-ODN;
iii. when the 3′-most region of the L-ODN anneals to the 3′-most region of the S-ODN, and the 5′-most region of the R-ODN anneals to the 5′-most region of the S-ODN, a gap is formed between the 3′ end of the L-ODN and the 5′ end of the R-ODN; and
iv. the I-ODN has a nucleotide sequence that allows it to anneal to a sequence of nucleotides termed the variable region of the S-ODN, where the gap is located across from the variable region.

In another aspect, the present invention provides a composition that includes a left oligonucleotide (L-ODN), a plurality of intermediate oligonucleotides (I-ODN), a right oligonucleotide (R-ODN) and a splint oligonucleotide (S-ODN), wherein
i. a 3′-most region of the L-ODN is functionally complementary to a 3′-most region of the S-ODN;
ii. a 5′-most region of the R-ODN is functionally complementary to a 5′-most region of the S-ODN;
iii. when the 3′-most region of the L-ODN anneals to the 3′-most region of the S-ODN, and the 5′-most region of the R-ODN anneals to the 5′-most region of the S-ODN, a gap is formed between the 3′ end of the L-ODN and the 5′ end of the R-ODN; and
iv. each I-ODN has a nucleotide sequence that allows it to anneal to a sequence of nucleotides termed the variable region of the S-ODN, where the gap is located across from the variable region; and
v. members of the plurality of I-ODNs have the same number of nucleotides but differ in their nucleotide sequences.

In another aspect, the present invention provides a composition that includes a left oligonucleotide (L-ODN), a plurality of intermediate oligonucleotides (I-ODNs), a right oligonucleotide (R-ODN) and a plurality of splint oligonucleotide (S-ODNs), wherein
i. a 3′-most region of the L-ODN is functionally complementary to a 3′-most region of the S-ODNs;
ii. a 5′-most region of the R-ODN is functionally complementary to a 5′-most region of the S-ODNs;
iii. when the 3′-most region of the L-ODN anneals to the 3′-most region of the S-ODNs, and the 5′-most region of the R-ODN anneals to the 5′-most region of the S-ODNs, a gap is formed between the 3′ end of the L-ODN and the 5′ end of the R-ODN; and
iv. each I-ODN has a nucleotide sequence that allows it to anneal to a sequence of nucleotides termed the variable region of the S-ODNs, where the gap is located across from the variable region;
v. members of the plurality of I-ODNs have the same number of nucleotides but differ in their nucleotide sequences; and
vi. members of the plurality of S-ODNs have the same number of nucleotides but differ in their nucleotide sequences within the variable region of the S-ODN.

In another aspect, the present invention provides a composition that includes a plurality of oligonucleotides, where each member of the plurality has a nucleotide sequence extending from a 3′ end to a 5′ end of the oligonucleotide, and
i. the nucleotide sequence of each oliognucleotide in the plurality consists of a R-ODN-derived sequence including the 3′-most nucleotide of the oligonucleotide, an I-ODN-derived sequence located between the R-ODN-derived sequence and the L-ODN-derived sequence, and a L-ODN-derived sequence including the 5′-most nucleotide of the oligonucleotide;
ii. the R-ODN-derived sequences of each member of the plurality are identical;
iii. the L-ODN-derived sequences of each member of the plurality are identical; and
iv. the I-ODN-derived sequences of each member of the plurality are different.

These and other aspects of the present invention are described in greater detail herein.

### BRIEF DESCRIPTION OF THE DRAWINGS

FIG. 1 illustrates a double-stranded polynucleotide having four components, namely, three polynucleotides (the L-ODN, the I-ODN (a hexamer) and the R-ODN) each hybridized to fourth polynucleotide (the S-ODN, having a length of 24 nucleotides).

FIG. 2 illustrates a double-stranded polynucleotide having four components, namely, three polynucleotides (the L-ODN, the I-ODN (a trimer) and the R-ODN) each hybridized to fourth polynucleotide (the S-ODN having a length of 24 nucleotides).

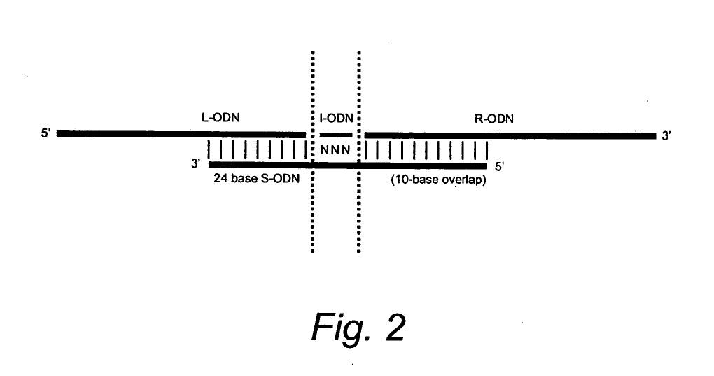

### DETAILED DESCRIPTION OF THE INVENTION

In a particularly useful aspect, the present invention provides a significantly improved synthetic method of producing a set of mutagenized progeny polynucleotides which contain at least one substituted codon encoding for each of the 20 naturally encoded amino acids or any selected subset thereof. This in turn, similarly provides a method for producing from a parental template polypeptide, a set of mutagenized progeny polypeptides in which each of the 20 naturally encoded amino acids is represented at each original amino acid position or any selected subset thereof. The methods described herein enable the synthesis of defined, complex mixtures of oligonucleotides, in instances where the incorporation of degenerate bases is impractical. These oligonucleotide mixtures are useful for a variety of applications such as recombination methods, site-saturation mutagenesis, or the like.

Prior to setting forth a more detailed description of the invention, the following definitions are provided as an aid to the reader's understanding of the detailed description and appended claims.

Definitions

The term “nucleic acid molecule” as used herein, is comprised of at least one base or one base pair, depending on whether it Is single-stranded (ss) or double-stranded (ds), respectively. A nucleic acid molecule may furthermore, belong exclusively or chimerically to any group of nucleotide-containing molecules, as exemplified by, but not limited to, the following groups of nucleic acid molecules: DNA, RNA, genomic nucleic acids, non-genomic nucleic acids, naturally and non-naturally occurring nucleic acids, and synthetic nucleic acids.

The term “amino acid” as used herein refers to any organic compound that contains an amino group (—NH₂) and a carboxyl group (—COOH); preferably either as free groups or alternatively after condensation as part of peptide bonds. The “twenty naturally encoded polypeptide-forming alpha-amino acids” are understood in the art and refer to: alanine (ala or A), arginine (arg or R), asparagines (asn or N), aspartic acid (asp or D), cysteine (cys or C), glutamic acid (glu or E), glutamine (gin or Q), glycine (gly or G), histidine (his or H), isoleucine (ile or I), leucine (leu or L), lysine (lys or K), methionine (met or M), phenylalanine (phe or F), proline (pro or P), serine (ser or S), threonine (thr or T), tryptophan (trp or W), tyrosine (tyr or Y), and valine (val or V).

The term “identical” or “identity” means that two nucleic acid sequences have the same sequence or a complementary sequence. Thus, “areas of identity” refers to regions or areas of a polynucleotide or the overall polynucleotide are identical or complementary to areas of another polynucleotide or the polynucleotide.

The terms “hybridize”, “anneal” or “binds to” refer to the ability of complementary (either exactly complementary or functionally complementary) DNA molecules (DNA1 and DNA2) to associate with one another and form Watson-Crick base pairs and/or heterocyclic base stacking interactions. It is understood by those versed in the art that “hybridization” or “binding” of one DNA molecule (or polynucleotide) to another means their affinity for one another is greater than their affinity to other, non-specific molecules. This affinity is present when DNA1 and DNA2 include exactly complementary sequences. In general, to determine whether exact complementarity is present in two DNA molecules (DNA1 and DNA2), one looks at the hybrid that forms between DNA1 and DNA2, and if all the base pairs that are present in this hybrid are standard Watson-Crick base pairs, then DNA1 and DNA2 have nucleotide sequences that are exactly complementary. Exact complementarity may be present in the DNA1/DNA2 pair even though DNA1 has nucleotides that do not form a base pair when DNA1 and DNA2 hybridize. This may occur, for example, when DNA1 has 20 nucleotides and DNA2 only has 10 nucleotides. In this case, the DNA1/DNA2 hybrid will form, at most, 10 base pairs, with 10 nucleotides of DNA1 being in unpaired form. However, so long at these 10 base pairs are each Watson-Crick base pairs, DNA1 and DNA1 have exactly complementary nucleotide sequences. As another example, each of DNA1 and DNA2 may have 20 nucleotides, however, the hybrid that forms between DNA1 and DNA2 results in base pairing between only the 15 contiguous nucleotides that form the 3′-most region of DNA1 and the 15 contiguous nucleotides that form the 5′ most region of DNA2. So long as these 15 base-paired nucleotides are paired according to Watson-Crick base-pairing rules, DNA1 and DNA2 have exactly complementary nucleotide sequences as this term is used herein.

“Specific hybridization” is defined herein as the formation of hybrids between a first polynucleotide and a second polynucleotide wherein substantially unrelated polynucleotide sequences do not form hybrids in the mixture. The term “complementary to” is used herein to mean that the complementary sequence is homologous to all or a portion of a reference polynucleotide sequence (in which G base pairs with C, and A base pairs with T).

The term “homologous” or “homeologous” means that one single-stranded nucleic acid sequence may hybridize to a complementary single-stranded nucleic acid sequence. The degree of hybridization may depend on a number of factors including the amount of identity between the sequences and the hybridization conditions such as temperature and salt concentrations. Preferably the region of identity is greater than about 5 bp, more preferably the region of identity is greater than 10 bp.

The term “mutation” means changes in the sequence of a parental nucleic acid sequence or changes in the sequence of a peptide. Such mutations may be point mutations such as transitions or transversions, or may also be deletions, insertions, or duplications.

The term “corresponds to” is used herein to mean that a polynucleotide sequence is homologous (i.e., is identical not strictly evolutionarily related) to all or a portion of a reference polynucleotide sequence, or that a polypeptide sequence is identical to a reference polypeptide sequence.

The term “degenerate nucleotide substitution” when used in reference to a set of polynucleotides denotes the situation where each member of the set has the same number of nucleotide residues (that number being denoted by “z”) and, with an indicated number of exceptions, the same nucleotide base sequence, where at each “exceptional” position the members of the set have A, G, C or T substitution, with each of these four options being represented by one member of the set. Thus, the number of exceptions indicates the number of members in the set according to the equation (number of exceptions) x 4=members in the set. Degenerate nucleotide substitution will be illustrated in the case where the number of exceptions is one, such that there are four polynucleotides (P1, P2, P3 and P4) in the set. Each of the polynucleotides P1, P2, P3 and P4 has the same nucleotide sequence except that the nucleotide in P1 that is a distance y nucleotides from the 3′ end of P1 is A, while the nucleotide in P2 that is the same distance (y) from the 3′ end of P2 is G, and the nucleotide that is located a distance y from the 3′ end of P3 is C, and the nucleotide that is located a distance y from the 3′ end of P4 is T. In other words, degenerate nucleotide substitution is present when four polynucleotides have the same base sequence except at a specific base, and in total these four polynucleotides have A, G, C, and T substitution at that specific base. The number z can be any integer of at least 2. The number y equals 1 when the nucleotide being referred to is the 3′-most nucleotide in the polynucleotide, and y equals z when the nucleotide being referred to is the 5′-most nucleotide in the polynucleotide. A set of polynucleotides may have degenerate nucleotide substitution at more than one nucleotide position. For example, a set of polynucleotides may have degenerate nucleotide substitution at location y and location y+1 (where location y+1 is adjacent to location y, and location y+1 is on the 5′ side of location y). In this case, the set consists of sixteen distinct polynucleotides, where each member of the set has substitution at nucleotide positions y/y+1 of either A/A, A/G, A/C, A/T, G/A, G/G, G/C, G/T, C/A, C/G, C/C, C/T, T/A, T/G, T/C, T/T, and each of these possible y/y+1 substitution patterns is present in one member of the set. Likewise, if a set of polynucleotides has degenerate nucleotide substitution at 3 nucleotide positions, then the set consists of 4×4×4=64 members. In an optional aspect of the invention, each member in the set is present at the same concentration in the composition that contains the set of polynucleotides. In other optional aspects, the members of the set are present in a composition at an average concentration of Δ (where Δ equals the concentration of the first member in the composition+the concentration of the second member in the composition, etc., divided by the number of members in the set), and each member of the set is present in the composition at a concentration of ±0.99Δ, or ±0.95Δ, or ±0.90Δ, or ±0.85Δ, or ±0.80Δ, or ±0.75Δ, or ±0.70Δ, or ±0.65Δ, or ±0.60Δ, or ±0.55Δ, or ±0.50Δ.

The term “isolated” is used to refer to the situation where something has been taken from, i.e., isolated, from its original environment. Optionally, the original environment is a natural environment if the “something” is naturally occurring. For example, a naturally-occurring polynucleotide or enzyme in a living animal is not isolated, but the same polynucleotide or enzyme, separated from some or all of the coexisting materials in the natural system, is isolated.

By “isolated nucleic acid” is meant a nucleic acid, e.g. a DNA or RNA molecule, that is not immediately contiguous with the 5′- or 3′-flanking sequences with which it normally is immediately contiguous when present In the naturally occurring genome of the organism from which it is derived. The term thus describes, for example, a nucleic acid that is incorporated into a vector, such as a plasmid or viral vector; a nucleic that is incorporated into the genome of a heterologous cell (or the genome of a homologous cell, but at a site different from that at which it naturally occurs); and a nucleic acid that exists as a separate molecule, e.g., a DNA fragment produced by PCR amplification or restriction enzyme digestion, or an RNA molecule produced by in vitro transcription. The term also describes a recombinant nucleic acid that forms part of a hybrid gene encoding additional polypeptide sequences that can be used, for example, in the production of a fusion protein.

“Ligation” refers to the process of linking two (or more) double-stranded DNA molecules or polynucleotides together, through the formation of internucleotide phosphodiester bonds. Unless specified otherwise, ligation may be accomplished using known buffers and conditions with 10 units of T4 DNA ligase (“ligase”) per 0.5 μg of approximately equimolar amounts of the DNA fragments to be ligated. For the invention disclosed herein, ligation may also refer to the formation of new phosphodiester bonds between two (or more) single-stranded DNA molecules or polynucleotides. In this context, one of the two strands contains “nicked single-stranded fragements,” and these DNA molecules are brought into close proximity by hybridization to a template molecule or “splint” which is complementary to a region of each of the single-stranded molecules to be linked via internucleotide phosphodiester bond formation.

The terms “nucleic acid sequence coding for” or a “DNA coding sequence of” or “nucleotide sequence encoding” a particular enzyme—as well as other synonymous terms—refer to a DNA sequence which is transcribed and translated into an enzyme when placed under the control of appropriate regulatory sequences. A “promoter sequence” is a DNA regulatory region capable of binding RNA polymerase in a cell and initiating transcription of a downstream (3′-direction) coding sequence. The promoter is part of the DNA sequence. This sequence region has a start codon at its 3′-terminus. The promoter sequence does include the minimum number of bases where elements necessary to initiate transcription at levels detectable above background. However, after the RNA polymerase binds the sequence and transcription is initiated at the start codon (3′-terminus with a promoter), transcription proceeds downstream in the 3′-direction. Within the promoter sequence will be found a transcription initiation site (conveniently defined by mapping with nuclease S1) as well as protein binding domains (consensus sequences) responsible for the binding of RNA polymerase.

The term “gene” means the segment of DNA involved in producing a polypeptide chain; it include regions preceding and following the coding region (leader and trailer) as well as intervening sequences (introns) between individual coding segments (exons).

The terms “polynucleotide” and “oligonucleotide” refer to polymers having deoxynucleotide residues as monomeric units. No distinction is made herein between the terms oligonucleotide and polynucleotide, even though the term oligonucleotide is frequently used in the art to refer to relatively short (e.g., less than 100 nucleotide) chemically-synthesized chains of nucleotide residues, while the term polynucleotide is commonly used in the art to refer to a nucleotide-derived polymer having more than about 100 nucleotides, typically made using biological reagents (e.g., enzymes). As these two terms are used herein, they both refer to a polymer chain comprising two or more deoxynucleotide residues.

Polynucleotides have two termini, i.e., the polymer chain has two ends, where these two termini are commonly distinguished from one another in the art by being denoted the 5′ end and the 3′ end, where this terminology is based on the direction in which the phosphate group of one nucleotide is joined to the hydroxyl group of the adjacent nucleotide. This terminology to identify the ends of a polynucleotide will be used herein. Furthermore, the term “3′ most region” and “5′ most region” will be used herein. The 3′-most region of a polynucleotide refers to the one or more contiguous nucleotide residues that include the nucleotide at the 3′ end of the polynucleotide. Likewise, the 5′-most region of a polynucleotide refers to the one or more contiguous nucleotide residues that include the nucleotide at the 5′ end of the polynucleotide. In various aspects of the present invention, a “region” optionally refers to 2, or 3, or 4, or 5, or 6, or 7, or 8, or 9, or 10, or 11, or 12, or 13, or 14, or 15, or 16, or 17, or 18, or 19, or 20, or 25, or 30, or 35, or 40, or 45, or 50, or 60, or 70, or 80, or 90, or 100 contiguous nucleotides.

However, as used herein, the terms “3′ end” and “5′ end” do not require any particular chemical group to be present at either the 3′ or 5′ end of the polynucleotide. In one aspect of the invention, a phosphate group is located at the 5′ end of the molecule, and a hydroxyl group is located at the 3′ end of the molecule.

A polynucleotide may be single-stranded, or may be annealed (also known as hybridized) to a polynucleotide having a complementary base sequence, so as to be in a double-stranded form. The term “complementary” as used herein has its ordinary meaning in the art, and refers to a base sequence in a first polynucleotide which, upon hybridization of the first polynucleotide to a second polynucleotide, forms A/T and G/C base pairs with nucleotides present in the second polynucleotide.

In certain aspects of the present invention, a region in one polynucleotide is complementary to a region in another polynucleotide. This is an important criteria because when this criteria is met, the two polynucleotides will hybridize to one another to provide a low energy, stable, double-stranded molecule.

An “oligonucleotide” (or synonymously an “oligo”) refers to either a single-stranded (ss) polydeoxynucleotide or two complementary polydeoxynucleotide strands that may be chemically synthesized. Such synthetic oligonucleotides may or may not have a 5′-phosphate. Those lacking a 5′-phosphate will not ligate to another oligonucleotide without adding a phosphate with an ATP in the presence of a kinase. A synthetic oligonucleotide will ligate to a fragment that has not been dephosphorylated.

Methods

In one aspect, the present invention provides a method for preparing a polynucleotide. This method includes combining at least four oliognucleotides to form a mixture, where these four oligonucleotides are denoted, for convenience, a left oligonucleotide (L-ODN), an intermediate oligonucleotide (I-ODN), a right oligonucleotide (R-ODN) and a splint oligonucleotide (S-ODN). These four oligonucleotides have nucleotide sequences that are related to one another in the following four ways:
i. the 3′-most region of the L-ODN consists of a nucleotide sequence that is functionally complementary to the nucleotide sequence that forms the 3′-most region of the S-ODN;
ii. the 5′-most region of the R-ODN consists of a nucleotide sequence that is functionally complementary to the nucleotide sequence that forms the 5′-most region of the S-ODN;
iii. the L-ODN and the R-ODN anneal to the S-ODN via their functionally complementary sequences to provide a gap of x nucleotides between the 3′ end of the L-ODN and the 5′ end of the R-ODN, where the gap is across from a contiguous sequence of nucleotides in the S-ODN which collectively forms the “variable region” of the S-ODN; and
iv. the I-ODN is formed from a number of nucleotides that is equal to the number of nucleotides that form the variable region of the S-ODN, and the I-ODN has a nucleotide sequence that is functionally complementary to the variable region of S-ODN.

When reference is made to a 3′-most region or a 5′-most region of an oligonucleotide, this is reference to a contiguous sequence of nucleotides that includes the nucleotide that is at the 3′ end of the oligonucleotide or the 5′ end of the oligonucleotide, respectively. A “region” may be, in various optional embodiments of the invention, described in terms of a minimum number of nucleotides, a maximum number of nucleotides, or both a minimum and maximum number of nucleotides. In various aspects of the invention, the minimum number of nucleotides present in a region is 2, or 3, or 4, or 5, or 6, or 7, or 8, or 9, or 10, or 11, or 12, or 13, or 14, or 15, or 16, or 17, or 18, or 19, or 20, or 21, or 22, or 23, or 24, or 25, or 26, or 27, or 28, or 29, or 30, or 31, or 32, or 33, or 34, or 35, or 36, or 37, or 38, or 39, or 40, or 45, or 50. In various aspects of the invention, the maximum number of nucleotides present in a region is 100, or 90, or 80, or 70, or 60, or 50, or 45, or 40, or 39, or 38, or 37, or 36, or 35, or 34, or 33, or 32, or 31, or 30, or 29, or 28, or 27, or 26, or 25, or 24, or 23, or 22, or 21, or 20, or 19, or 18, or 17, or 16, or 15, or 14, or 13, or 12, or 11, or 10, or 9, or 8, or 7, or 6, or 5. In various aspects of the invention, the number of nucleotides present in a region is described in terms of a range, where the minimum number of nucleotides present in the range is any of the above-identified minimum numbers 2-50, and the maximum number of nucleotides present in the range is any of the above-identified maximum numbers 100-5. In one aspect, a region has 5-15 nucleotides. When the number of nucleotides in two regions goes below about 5, then the hybrid formed between these two regions is not particularly stable. In general, the thermodynamic stability of a hybrid increases as the number of base pairs in the hybrid increases, and as the number of G+C base pairs in the hybrid increases. When a region has about 15 nucleotides, then it generally exhibits excellent thermodynamic stability for purposes of the method, regardless of the base sequence of the region. While regions of greater than 15 nucleotides are functionally suitable for use in the present method, the cost of preparing the oligonucleotides generally increases as the number of nucleotides in a region increases, and thus there is a financial disincentive to using needlessly long regions.

In addition to the number of nucleotides present in a region, a region may be described in terms of the degree to which it is complementary to a region in another oligonucleotide. In one aspect, the nucleotides present in a region of a first ODN are exactly complementary to the nucleotides present in a region of a second ODN. That is, when a hybrid forms between the first and second ODNs, standard Watson-Crick base pairs are formed between each nucleotide in the hybridizing region of the first ODN and each nucleotide in the hybridizing region of the second ODN.

However, the method of the present invention may be suitably conducted even though there is one or more mismatched bases in the hybridizing regions. For example, if a region consists of 15 nucleotides, so that the hybrid has 15 base pairs, it will typically be adequate for purposes of the invention if only 14 of those base pairs are standard Watson-Crick base pairs, and the 15^{th} base pair is mismatched, i.e., other than a standard Watson-Crick base pair. In this case, the two regions are “functionally complementary” because they contain a sufficient number of exactly complementary nucleotides to achieve the necessary location and stability of the hybrid for purposes of the invention. As will be appreciated by one of ordinary skill in the art, the number of mismatched base pairs that can be tolerated in a hybrid formed between two regions depends on many factors. For example, one mismatched base pair will have less impact on the stability and location specificity of a hybrid formed between fifteen nucleotides than it will on a hybrid formed between only five nucleotides. As another factor, mismatched base pairs can destabilize a hybrid to various extents depending on the size, shape, atomic constitution, etc. of the bases involved in the mismatch.

In general, in order to get the L-ODN to anneal with the S-ODN at a desired location, the region of the L-ODN that hybridizes to the region of the S-ODN in the hybrid is suitably about 5-15 nucleotides in length. Likewise, in order to get the R-ODN to anneal with the S-ODN at a desired location, the region of the R-ODN that hybridizes to the region of the S-ODN in the hybrid is suitably about 5-15 nucleotides in length. There is no particular connection between the number of nucleotides that base pair in the L-ODN+S-ODN hybrid and the number of nucleotides that base pair in the R-ODN+S-ODN hybrid, and therefore these numbers may be selected independently. In one aspect, the region of the L-ODN that hybridizes to the region of the S-ODN in the hybrid is 5-15 nucleotides in length, and the region of the R-ODN that hybridizes to the region of the S-ODN in the hybrid is 5-15 nucleotides in length. In one aspect, all of the base pairs in the hybrid are standard Watson-Crick base pairs. In another aspect, all but one of the base pairs in the hybrid are standard Watson-Crick base pairs. In another aspect, all but two of the base pairs in the hybrid are standard Watson-Crick base pairs.

The nucleotide sequences of the L-ODN and the R-ODN are typically selected in order to achieve a certain goal, e.g., the creation of a desired polynucleotide. In other words, the nucleotide sequences of the L-ODN and the R-ODN are typically fixed by the nature of the target desirably achieved by the person utilizing the method of the present invention. The primary function of the S-ODN is to hold the L-ODN and R-ODN in a desired relative orientation, such that there is a gap between the 3′ end of the L-ODN and the 5′ end of the R-ODN, where this gap will be filled by the I-ODN. Assuming that the nucleotide sequences of the L-ODN and the R-ODN are fixed by the desired goal of the method, a decision needs to be made concerning the length of the S-ODN and the nucleotides sequences of the regions of the S-ODN that will hybridize to the 3′-most region of the L-ODN and the 5′-most region of the R-ODN. As mentioned above, a region length of 5-15 nucleotides is typically adequate. As for the nucleotide sequence within each region, in one aspect of the invention, those sequences are exactly complementary to the 5-15 nucleotides that form the 3′-most region of the L-ODN and the 5-15 nucleotides that form the 5′-most region of the R-ODN. In another aspect of the invention, those sequences are functionally complementary to the 5-15 nucleotides that form the 3′-most region of the L-ODN and are functionally complementary to the 5-15 nucleotides that form the 5′-most region of the R-ODN. Nucleotide sequences that are functionally complementary achieve the same hybrids as do nucleotide sequences that are identically complementary, insofar as the same gap is created between the 3′-end of the L-ODN and the 5′-end of the R-ODN.

As mentioned above, the L-ODN, S-ODN and R-ODN are designed such that the L-ODN and the R-ODN anneal to the S-ODN via their functionally complementary sequences to provide a gap of x nucleotides between the 3′ end of the L-ODN and the 5′ end of the R-ODN, where the gap is across from a contiguous sequence of nucleotides in the S-ODN which collectively forms the “variable region” of the S-ODN. The nucleotide sequence of the S-ODN in the variable region should be designed with a view to the nucleotide sequence of the I-ODN. As also mentioned previously, the I-ODN is formed from a number of nucleotides that is equal to the number of nucleotides that form the variable region of the S-ODN, and the I-ODN has a nucleotide sequence that is functionally complementary to the variable region of S-ODN. The variable region of the S-ODN needs to be functionally complementary to the nucleotide sequence of the I-ODN because the I-ODN needs to be able to anneal to the S-ODN in a manner that fills the gap formed when the L-ODN and the R-ODN anneal to the S-ODN. That is, the L-ODN, the I-ODN and the R-ODN need to anneal to the S-ODN such that the 5′ end of the I-ODN is “ligatable to” the 3′ end of the L-ODN, and the 3′ end of the I-ODN is “ligatable to” the 5′ end of the R-ODN. In order for this to occur, the nucleotide sequence of the I-ODN must be functionally complementary to the nucleotide sequence of the variable region of the S-ODN. That is, one or more Watson-Crick base pairs need to form when the I-ODN hybridizes to the variable region of the S-ODN.

After the L-ODN, I-ODN, R-ODN and S-ODN are combined, the 3′ end of the L-ODN is ligated to the 5′ end of the I-ODN, and the 3′ end of the I-ODN (or, equivalently, the 3′ end of the ligation product that forms when the 3′ end of the L-ODN is ligated to the 5′ end of the I-ODN) is ligated to the 5′ end of the R-ODN. This forms a ligation product that comprises, from 5′ to 3′, the nucleotide sequence of the L-ODN, the nucleotide sequence of the I-ODN and the nucleotide sequence of the R-ODN.

In one aspect of the invention, the I-ODN is selected in order to achieve a nucleotide sequence in the ligation product that expresses a desired sequence of amino acids. That is, the I-ODN is selected in order to create a desired one or more codons in the ligation product, where the codons then are used to create a desired polypeptide.

In one aspect of the invention, a single L-ODN, a single R-ODN, a single I-ODN and a single S-ODN are combined to create a single ligation product. However, in another highly useful aspect of the present invention, a single L-ODN, a single R-ODN and a single S-ODN are combined with a family of I-ODNs (I-ODN1, I-ODN2, etc.) so as to create a family of ligation products, where each member of the family has one or more different nucleotide sequences at one or more particular locations due to incorporating a different I-ODN. In other words, the members of the family of I-ODNs differ in that they have, at the same relative location, a different sequence of one or more nucleotides that are referred to as the “variable region” of each I-ODN. According to this aspect of the invention, it will necessarily be the case that the hybrid which forms between the variable region of the S-ODN and the I-ODN cannot always be exactly complementary. However, in order to allow the hybrid to form, at least some nucleotides in the I-ODN must be exactly complementary to an equal number of nucleotides in the variable region of the S-ODN. This can be achieved by having a “fixed region” within the I-ODN, i.e., a sequence of one or more nucleotides that are identical in terms of compositions (A, G, C, T) and location within the members of the family of I-ODNs. For example, if a variable region of the family of I-ODNs has three nucleotides, I-ODN1 will have three nucleotides that form variable region (VR)1, and will also have nucleotides that are exactly complementary to a nucleotide sequence in the variable region of the S-ODN. I-ODN2 will look exactly like I-ODN1 (i.i., same number of nucleotides, same nucleotide sequence) except that, in lieu of the nucleotides that form VR1, I-ODN2 will have the nucleotides that form VR2. Likewise, I-ODN3 will exactly look like I-ODN1, except that in lieu of the nucleotides that form VR1, I-ODN3 will have the nucleotides that form VR 3. In this way, each I-ODN molecule will have at least one nucleotide region that is complementary to a portion of the variable region of the S-ODN (the fixed region, (FR) of the I-ODN), such that each I-ODN can anneal to this variable region of the S-ODN. As for the nucleotides of the variable region of the S-ODN that will correspond to the nucleotides in VR1, VR2, VR3 etc. in the family of I-ODNs, these can be selected with a view to being most amenable to mismatches. For instance, there are nucleotides known as “universal nucleotides” which can essentially hydrogen bond to any other nucleotide. Such universal nucleotides are a good choice for the nucleotides that will be present in the variable region of the S-ODN and which will need to correspond to the nucleotides of VRs 1, 2, 3, etc. of the I-ODNs.

Alternatively, one of the natural nucleotides, i.e., A, G, C or T can be placed in a location of the S-ODN that will correspond to the nucleotides of VRs 1, 2, 3, etc. In one aspect, the nucleotides that form VRs 1, 2, 3, etc. of each I-ODN are located between one or more nucleotides that will form standard Watson-Crick base pairs when the I-ODN hybridizes to the variable region of the S-ODN. In this way, the “mismatched” base pairs are sandwiched between matching base pairs when the hybrid is formed between each I-ODN and the S-ODN. Having standard Watson-Crick base pairs at each end of the I-ODN/S-ODN hybrid is helpful in allowing each end of the I-ODN to ligate to the appropriate end of the R-ODN and the L-ODN. However, while this is one option, it has been surprisingly found that it is not necessary to have the 5′ end of the I-ODN be in a Watson-Crick base pair with the variable region of the S-ODN in order for the 5′ end of the I-ODN to ligate to the 3′ end of the L-ODN. Likewise, it is not necessary that the 3′ end of the I-ODN be in a Watson-Crick base pair in order for this 3′ end to ligate to the 5′ end of the R-ODN.

In another highly useful aspect of the present invention, a single L-ODN and a single R-ODN are combined with a family of S-ODNs (S-ODN1, S-ODN2, etc.) and a family of I-ODNs (I-ODN1, I-ODN2, etc.) so as to create a family of ligation products, where each member of the family has a different sequence of nucleotides at a particular location due to incorporating a different I-ODN. In one embodiment of this aspect of the invention, the different S-ODNs will differ from one another only in the nucleotide(s) that base pair with the nucleotides present in the variable regions of the I-ODNs. When preparing the S-ODN molecules, it is very easy to create degenerate nucleotide substitution at one or more of the locations in the S-ODN that will base pair with a nucleotide that is in a variable region of the I-ODNs. In a preferred aspect of the invention, degenerate nucleotide substitution is present at those locations in the S-ODNs that will base pair with the nucleotides of the variable region of the I-ODNs.

Thus, in one aspect, the present invention provides a composition that includes 64 different oligonucleotides (these being S-ODNs), where each oligonucleotide has the same nucleotide sequence except at locations y, y+1 and y+2 from the 3′ end of the oligonucleotides, where the family has degenerate nucleotide substitution at each of locations y, y+1 and y+2 as measured from the 3′ end of the oligonucleotides. In various embodiments of this aspect of the invention, each of these 64 oligonucleotides has w nucleotides between y and the 3′ end of the oligonucleotide, and has v nucleotides between y+2 and the 5″ end of the oligonucleotide, where each of v and w is the same for each of the 64 oligonucleotides, however, each of v and w is independently characterized in terms of a minimum number of nucleotides, a maximum number of nucleotides, or a range of nucleotides where the range is defined by any one of the recited minimum number of nucleotides in combination with any one of the recited maximum number of nucleotides, where the minimum number of nucleotides is 0, or 1, or 2, or 3, or4, or 5, or 6, or 7, or 8, or 9, or 10, or 11, or 12, or 13, or 14, or 15, or 16, or 17, or 18, or 19, or 20, or 21, or 22, or 23, or 24, or 25, or 26, or 27, or 28, or 29, or 30, or 31, or 32, or 33, or 34, or 35, or 36, or 37, or 38, or 39, or 40, or 45, or 50, and the maximum number of nucleotides is 100, or 90, or 80, or 70, or 60, or 50, or 45, or 40, or 39, or 38, or 37, or 36, or 35, or 34, or 33, or 32, or 31, or 30, or 29, or 28, or 27, or 26, or 25, or 24, or 23, or 22, or 21, or 20, or 19, or 18, or 17, or 16, or 15, or 14, or 13, or 12, or 11, or 10, or9, or 8, or 7, or 6, or 5, or 4, or 3, or 2, or 1. In one aspect, each of v and w is selected from 5-15 nucleotides.

As mentioned above, the I-ODN is designed so that it anneals to the variable region of S-ODN in the gap. In one aspect of the invention, the I-ODN is x nucleotides in length, where x is an integer within the range of x¹-x², where x¹ is selected from the numbers 1, 2, 3, 4, 5, 6, 7, 8, 9, 10, 11, 12, 13, 14, 15, 16, 17, 18, 19 and 20, while x² is independently selected from the numbers 1, 2, 3, 4, 5, 6, 7, 8, 9, 10, 11, 12, 13, 14, 15, 16, 17, 18, 19 and 20, with the proviso that x² is greater than or equal to x¹, and when x¹ is equal to x², the x is not a range but simply a single integer.

As mentioned above, the method of the present invention includes combining a left oligonucleotide (L-ODN), one or a plurality of intermediate oligonucleotides (I-ODNs), a right oligonucleotide (R-ODN) and one or a plurality of splint oligonucleotides (S-ODNs) so as to form a mixture. While this mixture must contain at least one L-ODN, at least one R-ODN, at least one I-ODN and at least one S-ODN, the mixture may also contain other components, including other oligonucleotides (i.e., other oligonucleotides or polynucleotides). For example, when a method of the invention recites forming a mixture that contains “a” left oligonucleotide (L-ODN) where a 3′-most region of the L-ODN is complementary to a 3′-most region of the S-ODN, this does not preclude the inclusion in the mixture of one or more oligonucleotides that do not have the same exact nucleotide sequence as the L-ODN but which do have (in common with the L-ODN) a 3′-most region that is complementary to a 3′-most region of the S-ODN. While many other examples could be provided, it should suffice to say that a method which “includes” or “comprises” certain steps does not preclude additional steps being performed, and does not preclude addition components being included in a mixture formed by the method.

In a similar vein, the statement that a plurality of S-ODNs have the same number of nucleotides “but differ in their nucleotide sequences within the variable region of the S-ODN” indicates that a set of S-ODNs necessarily has the same number of nucleotides (e.g., 24), and each member has a string of nucleotides that form the “variable region”. While different members of the set will have different nucleotide sequences within the variable region, this is not to say that members of the set cannot have different nucleotide sequences at locations other than the variable region. However, when an oligonucleotide is described as “having” a range of nucleotides, e.g., 10-100, then this polynucleotide does not have 200 nucleotides even though a polynucleotide with 200 nucleotides does have 100 nucleotides.

As used herein, “a plurality” refers to two or more. In various optional aspects of the invention, “a plurality” may be replaced, independently at each occurrence, with any integer, including 2, or 3, or 4, or 5, or 6, or 7, or 8, or 9, or 10, or 11, or 12, or 13, or 14, or 15, or 16, or 17, or 18, or 19, or 20, or 21, or 22, or 23, or 24, or 25, or 26, or 27, or 28, or 29, or 30, or 31, or 32, or 33, or 34, or 35, or 36, or 37, or 38, or 39, or 40, or 41, or 42, or 43, or 44, or 45, or 46, or 47, or 48, or 49, or 50, or 51, or 52, or 53, or 54, or 55, or 56, or 57, or 58, or 59, or 60, or 61, or 62, or 63, or 64, etc.

Compositions

In various aspects, the present invention provides compositions that are useful in, and prepared according to, the methods of the present invention. For instance, in one aspect, the present invention provides a composition comprising a left oligonucleotide (L-ODN), an intermediate oligonucleotide (I-ODN), a right oligonucleotide (R-ODN) and a splint oligonucleotide (S-ODN), wherein
i. a 3′-most region of the L-ODN is functionally complementary to a 3′-most region of the S-ODN;
ii. a 5′-most region of the R-ODN is functionally complementary to a 5′-most region of the S-ODN;
iii. when the 3′-most region of the L-ODN anneals to the 3′-most region of the S-ODN, and the 5′-most region of the R-ODN anneals to the 5′-most region of the S-ODN, a gap is formed between the 3′ end of the L-ODN and the 5′ end of the R-ODN; and
iv. the I-ODN has a nucleotide sequence that allows it to anneal to a sequence of nucleotides termed the variable region of the S-ODN, where the gap is located across from the variable region.

In another aspect, the present invention provides a composition comprising a left oligonucleotide (L-ODN), a plurality of intermediate oligonucleotides (I-ODN), a right oligonucleotide (R-ODN) and a splint oligonucleotide (S-ODN), wherein
i. a 3′-most region of the L-ODN is functionally complementary to a 3′-most region of the S-ODN;
ii. a 5′-most region of the R-ODN is functionally complementary to a 5′-most region of the S-ODN;
iii. when the 3′-most region of the L-ODN anneals to the 3′-most region of the S-ODN, and the 5′-most region of the R-ODN anneals to the 5′-most region of the S-ODN, a gap is formed between the 3′ end of the L-ODN and the 5′ end of the R-ODN; and
iv. each I-ODN has a nucleotide sequence that allows it to anneal to a sequence of nucleotides termed the variable region of the S-ODN, where the gap is located across from the variable region; and
v. members of the plurality of I-ODNs have the same number of nucleotides but differ in their nucleotide sequences.

In yet another aspect, the present invention provides a composition comprising a left oligonucleotide (L-ODN), a plurality of intermediate oligonucleotides (I-ODNs), a right oligonucleotide (R-ODN) and a plurality of splint oligonucleotide (S-ODNs), wherein
i. a 3′-most region of the L-ODN is functionally complementary to a 3′-most region of the S-ODNs;
ii. a 5′-most region of the R-ODN is functionally complementary to a 5′-most region of the S-ODNs;
iii. when the 3′-most region of the L-ODN anneals to the 3′-most region of the S-ODNs, and the 5′-most region of the R-ODN anneals to the 5′-most region of the S-ODNs, a gap is formed between the 3′ end of the L-ODN and the 5′ end of the R-ODN; and
iv. each I-ODN has a nucleotide sequence that allows it to anneal to a sequence of nucleotides termed the variable region of the S-ODNs, where the gap is located across from the variable region;
v. members of the plurality of I-ODNs have the same number of nucleotides but differ in their nucleotide sequences; and
vi. members of the plurality of S-ODNs have the same number of nucleotides but differ in their nucleotide sequences within the variable region of the S-ODN.

In another aspect, the present invention provides compositions that include a plurality of polynucleotides, where these compositions may be prepared by the ligation method described herein. Each member of the plurality can be described by its nucleotide sequence which extends from the 3′ end to the 5′ end of the subject polynucleotide. As these compositions may be prepared by the methods of the invention as described herein, the nucleotide sequence of each polynucleotide in the plurality may be described in terms of having a R-ODN-derived nucleotide sequence that includes the 3′-most nucleotide of the oligonucleotide, an I-ODN-derived nucleotide sequence located between the R-ODN-derived sequence and the L-ODN-derived sequence, and a L-ODN-derived nucleotide sequence that includes the 5′-most nucleotide of the oligonucleotide. The I-ODN-derived nucleotide sequence will preferably have at least one variable region (VR) and at least one fixed region (FR), where the fixed region has the same nucleotide sequence in all of the members of the plurality, while the nucleotide sequence in the variable region is different between members of the plurality. The R-ODN-derived sequences of each member of the plurality are identical, i.e., each member of the plurality has the same R-ODN-derived nucleotide sequence. In addition, the L-ODN-derived nucleotide sequences of each member of the plurality are identical, i.e., each member of the plurality has the same L-ODN-derived nucleotide sequence. In fact, the only difference between members of the plurality lies in having different I-ODN-derived nucleotide sequences, and more specifically, in having different nucleotide sequences (relative to other members of the plurality) within each of the one or more variable regions of the I-ODN-derived nucleotide sequences.

The following criteria may be used, alone or in any combination, to further describe these compositions that include a plurality of polynucleotides:
the plurality has 2, or 3, or 4, or 5, or 6, or 7, or 8, or 9, or 10, or 11, or 12, or 13, or 14, or 15, or 16, or 17, or 18, or 19, or 20, or 21, or 22, or 23, or 24, or 25, or 26, or 27, or 28, or 29, or 30, or 31, or 32, or 33, or 34, or 35, or 36, or 37, or 38, or 39, or 40, or 41, or 42, or 43, or 44, or 45, or 46, or 47, or 48, or 49, or 50, or 51, or 52, or 53, or 54, or 55, or 56, or 57, or 58, or 59, or 60, or 61, or 62, or 63, or 64, members;
the plurality comprises 2, or 3, or 4, or 5, or 6, or 7, or 8, or 9, or 10, or 11, or 12, or 13, or 14, or 15, or 16, or 17, or 18, or 19, or 20, or 21, or 22, or 23, or 24, or 25, or 26, or 27, or 28, or 29, or 30, or 31, or 32, or 33, or 34, or 35, or 36, or 37, or 38, or 39, or 40, or 41, or 42, or 43, or 44, or 45, or 46, or 47, or 48, or 49, or 50, or 51, or 52, or 53, or 54, or 55, or 56, or 57, or 58, or 59, or 60, or 61, or 62, or 63, or 64, members;
the R-ODN-derived nucleotide sequence is at least 1, or 2, or 3, or 4, or 5, or 6, or 7, or 8, or 9, or 10, or 11, or 12, or 13, or 14, or 15, or 16, or 17, or 18, or 19, or 20, or 21, or 22, or 23, or 24, or 25, or 26, or 27, or 28, or 29, or 30, or 31, or 32, or 33, or 34, or 35, or 36, or 37, or 38, or 39, or 40, or 45, or 50 nucleotides in length;
the R-ODN-derived nucleotide sequence is not more than 100, or 90, or 80, or 70, or 60, or 50, or 45, or 40, or 39, or 38, or 37, or 36, or 35, or 34, or 33, or 32, or 31, or 30, or 29, or 28, or 27, or 26, or 25, or 24, or 23, or 22, or 21, or 20, or 19, or 18, or 17, or 16, or 15, or 14, or 13, or 12, or 11, or 10, or 9, or 8, or 7, or 6, or 5, or 4, or 3, or 2, or 1 nucleotides in length;
the L-ODN-derived nucleotide sequence is at least 1, or 2, or 3, or 4, or 5, or 6, or 7, or 8, or 9, or 10, or 11, or 12, or 13, or 14, or 15, or 16, or 17, or 18, or 19, or 20, or 21, or 22, or 23, or 24, or 25, or 26, or 27, or 28, or 29, or 30, or 31, or 32, or 33, or 34, or 35, or 36, or 37, or 38, or 39, or 40, or 45, or 50 nucleotides in length;
the L-ODN-derived nucleotide sequence is not more than 100, or 90, or 80, or 70, or 60, or 50, or 45, or 40, or 39, or 38, or 37, or 36, or 35, or 34, or 33, or 32, or 31, or 30, or 29, or 28, or 27, or 26, or 25, or 24, or 23, or 22, or 21, or 20, or 19, or 18, or 17, or 16, or 15, or 14, or 13, or 12, or 11, or 10, or 9, or 8, or 7, or 6, or 5, or 4, or 3, or 2, or 1 nucleotides in length;
the I-ODN-derived nucleotide sequence is at least 1, or 2, or 3, or 4, or 5, or 6, or 7, or 8, or 9, or 10, or 11, or 12, or 13, or 14, or 15, or 16, or 17, or 18, or 19, or 20, or 21, or 22, or 23, or 24, or 25, or 26, or 27, or 28, or 29, or 30, or 31, or 32, or 33, or 34, or 35, or 36, or 37, or 38, or 39, or 40, or 45, or 50 nucleotides in length;
the I-ODN-derived nucleotide sequence is not more than 100, or 90, or 80, or 70, or 60, or 50, or 45, or 40, or 39, or 38, or 37, or 36, or 35, or 34, or 33, or 32, or 31, or 30, or 29, or 28, or 27, or 26, or 25, or 24, or 23, or 22, or 21, or 20, or 19, or 18, or 17, or 16, or 15, or 14, or 13, or 12, or 11, or 10, or 9, or 8, or 7, or 6, or 5, or 4, or 3, or 2, or 1 nucleotides in length; and
the I-ODN-derived nucleotide sequence has a number p contiguous nucleotides (“the fixed region”) that are identically present in each member of the plurality, and a number q contiguous nucleotides (“the variable region”) that are different in each member of the plurality, where the I-ODN-derived nucleotide sequence has, in total, p+q nucleotides, and in various embodiments p is any one or more of 1, or 2, or 3, or 4, or 5, or 6, or 7, or 8, or 9, or 10, or 11,or 12, or 13, or 14, or 15, or 16, or 17, or 18, or 19, or 20, or 21, or 22, or 23, or 24, or 25, or 26, or 27, or 28, or 29, or 30, or 31, or 32, or 33, or 34, or 35, or 36, or 37, or 38, or 39, or 40, or 45, or 50 nucleotides, and independently q is any one or more of 1, or 2, or 3, or 4, or 5, or 6, or 7, or 8, or 9, or 10, or 11, or 12, or 13, or 14, or 15, or 16, or 17, or 18, or 19, or 20, or 21, or 22, or 23, or 24, or 25, or 26, or 27, or 28, or 29, or 30, or 31, or 32, or 33, or 34, or 35, or 36, or 37, or 38, or 39, or 40, or 45, or 50 nucleotides. Preferably, q is three nucleotides, because this is the number of nucleotides that encodes for an amino acid, and the reading frame for the product polynucleotide when it is inserted into a translation system (i.e., a system that converts the information encoded in a polynucleotide into a polypeptide structure) will recognize these three nucleotides as a single codon. However, q could be two nucleotides that were included within a single codon according to the reading frame that translates the polynucleotide, in which case one nucleotide adjacent to the q nucleotides would be constant, i.e., each codon that includes the two q nucleotides would either terminate or begin with the constant nucleotide. Alternatively, q could be two nucleotides that were ultimately present in different codons according to the reading frame used to translate the polynucleotide, in which case these two adjacent codons will each have two constant nucleotides and one variable nucleotide (the q nucleotide). As can be seen, the method of the present invention provides tremendous control in producing a family of polynucleotides that encodes exactly the variability that is desired.

The methods of the present invention allow for the preparation of compositions that contain a plurality of polynucleotides, where the members either do, or do not, have any particular nucleotide sequence, within the one or more variable regions of the I-ODN-derived nucleotide sequence. For example, when a variable region is formed from three nucleotides, various aspects of the invention provide compositions wherein one member has any selected one of the following sequences within a particular variable region in a I-ODN-derived nucleotide sequence, or two members have any selected two of the following sequences within their particular variable regions in a I-ODN-derived nucleotide sequence, or three members have any selected three of the following sequences within their variable regions in a I-ODN-derived nucleotide sequence, etc. for 4 members, 5 members, 6 members, etc., independent of the number of members in the plurality, although of course the plurality must have at least as many members as are specified to have one of the following sequences (i.e., if the composition is specified to comprise 4 members, each having a selected one of the following sequences, then of course the plurality must include at least four members, however that plurality may include additional members) where the following sequences are written in the 5′→3′ direction: AAA, AAG, AAC, AAT, AGA, AGG, AGC, AGT, ACA, ACG, ACC, ACT, ATA, ATG, ATC, ATT, GAA, GAG, GAC, GAT, GGA, GGG, GGC, GGT, GCA, GCG, GCC, GCT, GTA, GTG, GTC, GTT, CAA, CAG, CAC, CAT, CGA, CGG, CGC, CGT, CCA, CCG, CCC, CCT, CTA, CTG, CTC, CTT, TAA, TAG, TAC, TAT, TGA, TGG, TGC, TGT, TCA, TCG, TCC, TCT, TTA, TTG, TTC, and TTT.

Likewise, the members of the plurality of polynucleotides in the compositions of the invention may be described as not having a selected one or more of the following nucleotide sequences within one or more specified variable region(s) in a I-ODN-derived nucleotide sequence of the polynucleotide. In other words, in addition to, or instead of, specifying what sequences necessarily are represented within the variable region(s) of the I-ODN-derived nucleotide sequence of a member of the plurality, the compositions of the present invention may be described in terms of what sequences are necessarily not represented within the variable region(s) of the I-ODN-derived nucleotide sequence of members of the plurality. Those nucleotide sequences that may be missing from the specified variable region of the plurality are any one or more of (where sequences are written in the 5′→3′ direction): AAA, AAG, AAC, AAT, AGA, AGG, AGC, AGT, ACA, ACG, ACC, ACT, ATA, ATG, ATC, ATT, GAA, GAG, GAC, GAT, GGA, GGG, GGC, GGT, GCA, GCG, GCC, GCT, GTA, GTG, GTC, GTT, CAA, CAG, CAC, CAT, CGA, CGG, CGC, CGT, CCA, CCG, CCC, CCT, CTA, CTG, CTC, CTT, TAA, TAG, TAC, TAT, TGA, TGG, TGC, TGT, TCA, TCG, TCC, TCT, TTA, TTG, TTC, and TTT.

Thus, the compositions of the present invention are unique in that they can provide exactly the desired nucleotides or nucleotide sequences within the variable region(s) of the I-ODN-derived region of the polynucleotides, and do not provide any undesired nucleotides or nucleotide sequences within the variable region(s) of the I-ODN-derived region of the polynucleotides. This level of control in the preparation of a plurality of polynucleotides is not available in known methods for generating compositions useful in site saturation mutagenesis. For instance, in various aspects of the invention, the composition contains exactly t member polynucleotides, where these t members each have the same nucleotide sequence except, within the or a variable region of the I-ODN-derived polynucleotide sequence, each of the t members has a three-nucleotide sequence (a codon) that encodes for a different one of the t natural amino acids, where t is an integer of 1, 2, 3, 4, 5, 6, 7, 8, 9, 10, 11, 12, 13, 14, 15, 16, 17, 18, 19 or 20.

More specifically, and as one example, both of codons TTT and TTC encode for the amino acid phenylalanine. In one aspect of the invention, only one codon encoding for a specific amino acid is present within a particular variable region of the polynucleotides in the composition. Thus, only one of TTT or TTC would be present among the plurality of polynucleotides in the composition of the invention at a particular variable region. Now, as mentioned earlier, the I-ODN-derived nucleotide sequence may have more than one variable region, and more than one fixed region. In the compositions comprising a plurality of polynucleotides according to the invention, there is only variation in nucleotide sequences within the variable region(s) among/between the members. While the “fixed regions”, if present, will also ultimately encode one or more amino acids, these fixed regions will all encode the same amino acids among the plurality of members. Thus, the statement that only one TTT or TTC would be present among the plurality of polynucleotides in the compositions, refers to the presence of these sequences within the variable regions—of course, either one of these sequences may be present in the variable region and the other sequence could be present in a fixed region, or with the nucleotide sequences that are termed the L-ODN-derived nucleotide sequence or the R-ODN-derived nucleotide sequence.

It should also be clarified that the I-ODN-derived nucleotide sequence may have more than one variable region and, independently, more than one fixed region. For instance, the I-ODN-derived nucleotide sequence may have a fixed region, a variable region, and then another fixed region, as viewed from the 5′→3′ direction of the I-ODN, where this may be abbreviated as a 1^{st}F-V-2^{nd}F sequence. This may be useful because ligation between the I-ODN and the R-ODN can then occur between nucleotides that are base-paired according to standard Watson-Crick base pairing rules, while ligation between the I-ODN and the L-ODN can also occur between nucleotides that are base-paired according to standard Watson-Crick base pairing rules. As another alternative, the I-ODN-derived nucleotide sequence may be formed from a first variable region, a second variable region, and a fixed region, again as viewed from the 5′→3′ direction, where this may be abbreviated as a 1^{st}V-2^{nd}V-F sequence. In this way, a family of polynucleotides is created having some variability in the nucleotide sequence at the first variable region, and independently having some variability in the nucleotide sequence at the second variable region. Any other arrangement of codon and variable regions within the I-ODN-derived nucleotide sequence is possible, including, as viewed from the 5′→3′ direction, F-V; V-F; F-V-F, V-F-V, V-V-F, F-V-V, where, as stated previously each F or V region may independently have, for example, 1, or 2, or 3, or 4, or 5, or 6, or 7, or 8, or 9, or 10, or 11, or 12, or 13, or 14, or 15, or 16, or 17, or 18, or 19, or 20, or 21, or 22, or 23, or 24, or 25, or 26, or 27, or 28, or 29, or 30, or 31, or 32, or 33, or 34, or 35, or 36, or 37, or 38, or 39, or 40, or 45, or 50 nucleotides.

Other components that may be present in the compositions comprising a plurality of polynucleotides that are formed according to method of the present invention. For instance, the composition may include the S-ODN molecule(s) that were used to form the plurality of polynucleotides. In addition, or instead, the composition may include a ligase that was used to form the plurality of polynucleotides.

Solid Supports

The methods of the present invention may be performed on a solid support. For instance, the L-ODN, the R-ODN, one or more I-ODN, or one or more S-ODN may be bound to a solid support, when conducting a method of the present invention. Having an oligonucleotide bound to a solid support is particularly useful from a purification point of view, because bound oligonucleotides, and oligonucleotides hybridized to bound oligonucleotides, are readily separated from oligonucleotides and other materials that are in solution. Methods to bind an oligonucleotide to a solid support, including methods of preparing an oligonucleotide bound to a solid support, are well known in the art and may be used in the present invention.

### Some Exemplary Embodiments

One embodiment of the present invention utilizes a strategy in which two unique oligonucleotide fragments (L-ODN and R-ODN) and a defined set of pooled oligonucleotide hexamers (i.e., a family of I-ODNs) are combined and ligated to afford the desired mixed set of oligonucleotides. In this context, a hexameric I-ODN may represent two codons that code for consecutive amino acids. A combinatorial pooled ligation strategy that enables specific insertions to be made of all 20 amino acid codons (or a subset thereof), in a convergent, one-pot synthesis methodology as provided by the present invention is extremely useful. The ligation reactions all proceed to a similar extent of completion since each reaction is template driven (i.e., facilitated by a partially complementary region within S-ODN), thereby providing the defined complex mixture without biases due to base composition of the inserted hexamers.

In one aspect of the invention, a library of 400 hexamers is generated and inventoried that consists of 20 different trinucleotides (representing all 20 amino acid codons) and an adjacent set of the 20 trinucleotides (similarly representing all 20 codons for the next contiguous three base subunits). Hence, only three oligonucleotides need to be synthesized (the L-ODN, the R-ODN and the S-ODN, although the S-ODN may have degenerate substitution in the variable region), not counting the hexamer library, in order to produce a given set of mixed oligonucletides. An appropriate set of hexamers may be selected based on various criteria, including codon usage frequency (see, e.g., E. coli K12 usage frequency table, (see, e.g., www.kazusa or jp/codon/)), base composition (attempting to balance the GC-content to minimize biases with the combinatorial splint during the ligation reaction, see below), and sequence screening in order to minimize and/or preclude self-hybridization, i.e., palindromes, whenever possible (to ensure that each hexamer is similarly available to associate with the combinatorial splint and react in the pooled ligation).

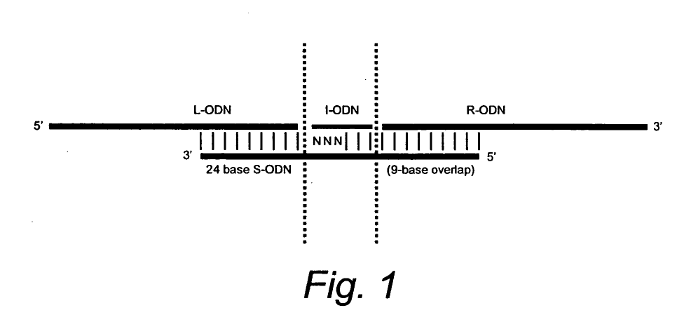

A preferred embodiment of the invention is to synthesize a set of oligonucleotide primers for site-saturated mutagenesis, whereby two synthetic oligonucleotides (representing the conserved left and right flanks of the desired primer mixture, i.e., the L-ODN and R-ODN molecules) are mixed with a given set of hexamers (i.e., a family of I-ODN molecules) and a complementary combinatorial oligonucleotide splint (i.e., a family of S-ODN molecules having degenerate nucleotide substitution in the variable region), used to facilitate the template-driven ligation reaction. This mixture is ligated (enzymatically or chemically) to afford the desired mixed set of full-length oligonucleotides. The synthetic splint (S-ODN) is used to facilitate hybridization so that the left- and right-flank primers are in close proximity to, and properly aligned with, the set of hexamers to be inserted in the ligation reaction(s). The splint is a combinatorial synthetic mixture which is complementary to a portion of both the adjacent conserved oligonucleotide flanks, the fixed trinucleotide codon in which to be inserted, and also contains either random and/or universal bases opposite the variable trinucleotide codon to be inserted in the ligation reaction(s), as depicted below in FIG. 1. Surprisingly, even though the exact complement within the combinatorial splint represents <5% for each hexamer, the insertion of each hexamer goes nearly to completion.

In a second preferred embodiment of the invention trimers are used in place of the hexamers in the first preferred embodiment to synthesize a set of oligonucleotide primers for site-saturated mutagenesis. As in the first embodiment, two synthetic oligonucleotides (representing the conserved left and right flanks of the desired primer mixture, i.e., the L-ODN and R-ODN molecules) are mixed with a given set of trimers and a complementary combinatorial oligonucleotide splint. This mixture is ligated to afford the desired mixed set of full-length oligonucleotides. The synthetic splint is used to facilitate hybridization so that the left- and right-flank primers are in close proximity to, and properly aligned with, the set of trimers to be inserted in the ligation reaction(s). The splint is a combinatorial synthetic mixture that is complementary to a portion of both the adjacent conserved oligonucleotide flanks, and contains either random and/or universal bases opposite the vanable trinucleotide codon to be inserted in the ligation reaction(s). Surprisingly, even though the complement of the trimer is present only in the random or universal bases, the insertion of each trimer ligates efficiently to form a full-length set of molecules, each composed of an L-ODN, a trimer, and an R-ODN.

Following the pooled ligation reaction the full-length oligonucleotide combinatorial pool (containing either hexamers or trimers, depending on the embodiment of the invention) can be conveniently purified (and the combinatorial splint removed) via HPLC, to isolate the desired mixture of purified, single-stranded oligonucleotide primers.

The following examples are illustrative of methods and compositions of the present invention, and are not intended to be a limitation thereon.

### EXAMPLES

### Example 1

Synthesis of a Codon-varied Oligonucleotides Using Ligase With a Complementary Splint

The initial set of experiment was to ligate a series of individual hexamers between the left- and right-flank primers using a splint that is perfectly complementary to each individual hexamer. The product from these reactions are compared by HPLC analysis with authentic chemically synthesized full-length oligonucleotide. The first six ligation reactions are listed below. The desired full-length oligonucleotide in each reaction is the 46mer product formed from the 24mer left-flank primer (L-ODN), the hexamer insert in the center, and the 16mer right-flank primer (R-ODN). The splint molecules utilized in this study complement the 10 bases at the 3′-end of the L-ODN, all six bases of each hexamer, and the contiguous 10 bases at the 5′-end of the R-ODN.

| | |
|---|---|
| Ala-Phe-Primer Rxn. 1-1: | |
| 5′-[CATAAGGATGGCC CACCAATTTC] GCGTTC [ATCTGCCGAT CAGGG] | (SEQ ID NOS: 1-3) respectively |
| Ala-Phe Splint = 3′-GTGGTTAAAGCGCAAGTAGACGGCTA | |
| Arg-Phe-Primer Rxn. 1-2: | |
| 5′-[CATAAGGATGGCC CACCAATTTC] CGCTTC [ATCTGCCGAT CAGGG] | (SEQ ID NOS: 4-6) respectively |
| Arg-Phe Splint = 3′-GTGGTTAAAGGCGAAGTAGACGGCTA | |
| Asn-Phe- Primer Rxn. 1-3: | |
| 5′-[CATAAGGATGGCC CACCAATTTC] AACTTC [ATCTGCCGAT CAGGG] | (SEQ ID NO: 7-9) respectively |
| Asn-Phe Splint = 3′-GTGGTTAAAGTTGAAGTAGACGGCTA | |
| Asp-Phe-Primer Rxn. 1-4: | |
| 5′-[CATAAGGATGGCC CACCAATTTC] GATTTC [ATCTGCCGAT CAGGG] | (SEQ ID NOS: 10-12) respectively |
| Asp-Phe Splint = 3′-GTGGTTAAAGCTAAAGTAGACGGCTA | |
| Cys-Phe-Primer Rxn. 1-5: | |
| 5′-[CATAAGGATGGCC CACCAATTTC] TGCTTC [ATCTGCCGAT CAGGG] | (SEQ ID NOS: 13-15) respectively |
| Cys-Phe Splint = 3′-GTGGTTAAAGACGAAGTAGACGGCTA | |
| Gln-Phe-Primer Rxn. 1-6: | |
| 5′-[CATAAGGATGGCC CACCAATTTC] CAGTTC [ATCTGCCGAT CAGGG] | (SEQ ID NOS: 16-18) respectively |
| Gln-Phe Splint = 3′-GTGGTTAAAGGTCAAGTAGACGGCTA | |

Oligonucleotides were prepared using an ABI 3900 DNA Synthesizer from Applied Biosystems, Inc. (Foster City, Calif., USA) with ABI Synthesis Columns using synthesis cycles provided by the vendor. All standard phosphoramidites and ancillary synthesis reagents are obtained from Glen Research, Inc. (Sterling, Va., USA). Concentrated ammonia and synthesis grade acetonitrile are obtained from Fisher Scientific (Springfield, N.J., USA). After ammonia treatment, the synthesized oligonucleotides are evaporated to dryness in a SpeedVac (Savant, Farmingdale, N.Y.) and resuspended in HPLC grade water. Concentrations of the oligonucleotides are determined by reading the 260 nm absorbance in 20 mM Tris, pH 7, on a Pharmacia LKB Ultrospec III (Amersham Pharmacia, Upsala, Sweden).

All oligonucleotides were diluted to a working concentration of 75 pmol/μL and kinased as follows. To 53 μL of oligo (4000 pmol) was added 20 μL of kinase mix and 27 μL of water. The mixture was then subjected to standard kinase cycle on an MJ Thermocycler. The kinase was denatured by heating at 65° C. for 10 minutes. The kinased oligonucleotides were used as is with no further purification. The oligonucleotides are used to form duplex fragments by drying 500 pmoles each of the complementary oligonucleotides in a speedvac and resuspending in 10 μL TE. A 5 μL sample of the solution (250 pmoles) is mixed with 10 microliters of 2×SSPE (prepared according to Manniatis).

Duplexes are successively ligated together to make longer fragments until the full length product is made. Each ligation consists of 500 picomoles of a pair of double-stranded oligonucleotide, 3 μL of 10× ligation buffer (Fermentas Inc., Hanover, Md.), 10 units of T4 DNA ligase (product #EL0016, Fermentas) and water to make a total volume of 30 μL. All duplexes are ligated together under the same conditions. Each ligation mix is incubated at 37° C. for 60 minutes, heated to 65° C. for 10 minutes and the fragment isolated by HPLC.

Next, “nicked” duplexes were formed from the kinased oligos (L-ODN, kinased hexameric I-ODN, and kinased R-ODN) and the perfectly matched splint as follows. To 43 μL of L-ODN (3200 pmol) was added, 80 μL of kinased hexamer (3200 pmol), 80 μL of kinased R-ODN (3200 pmole), and 47 μL of the perfectly matched complementary splint (S-ODN, 3525 pmol) for a total volume of 250 μL to form 12.8 pmol/μL of a“gapped” duplex. This was heated to 95° C. and cooled to room temperature.

The nicked duplex formed above was ligated as follows. To 50 μL of the nicked duplex (≧600 pmol), was added 10 μL of a ligation mixture composed of T4 DNA ligase and ligase buffer, both from Fermentas Inc. (Hanover, Md., USA, @fermantes.com). The reaction mixture was incubated at 4° C. for 24 hours, the ligase was heat kill denatured at 65° C. for 10 minutes, and the products were directly analyzed by HPLC.

High performance liquid chromatography (HPLC) is performed on a ProStar Helix HPLC system from Varian Inc. (Walnut Creek, Calif., USA) consisting of two high-precision high-pressure pumps (ProStar 215 Solvent Delivery Modules), a microtiter plate autosampler (ProStar 430 autosampler), a column oven (ProStar 510 Air Oven), a UV detector (ProStar 320 UV/Vis Detector) and a fraction collector (Dynamax FC-1 Fraction Collector), all controlled by Star Chromatography Workstation Software (Version 5.31). The column used is a Waters Xterra MS C₁₈ Column (4.6 mm ID×50 mm, 2.5 micron) from Waters Corp. (Milford, Mass., USA), and the following buffers were prepared: Buffer A: 5% acetonitrile in 100 mM triethylammonium acetate, pH 7.0 and Buffer B: 15% acetonitrile in 100 mM triethylammonium acetate, pH 7.0. The buffers were made from 2M triethylammonium acetate from Glen Research (Sterling, Va., USA, @glenres.com)and HPLC grade acetonitrile from Fisher Scientific.

The thermal and gradient conditions for isolating and analyzing the oligonucleotides and ligation reactions were a gradient of 20% to 57.5% buffer B in 15 minutes at 1 mL/min at 60° C. The sample was monitored at 260 nm. Preparative samples were collected based on peak shape and concentrated with ultracentrifugation using Microcon YM-3 centricon from Millipore, Inc. (Milford, Mass., USA). The retentate was collected in 25 mL of water. The OD at 260 nm was determined by diluting the retentate 1 to 40 dilution in 20 mM Tris, pH 7.0.

### Example 2

HPLC Comparison of Codon-varied Oligonucleotides Made Using Ligase and a Complementary Splint

HPLC conditions for the comparison of analysis was used to compare the products formed by ligation to the full-length synthetic primer standards, as well as the products from a pooled ligation of the six ligation reactions shown above. Retention times of the ligation products were indistinguishable from the full-length synthetic standards, and this was further confirmed with a series of HPLC coinjections.

### Example 3

Single Ligation Reaction Synthesis of Codon-varied Oligonucleotides Pools Using a 3/6 Combinatorial Splint

The next set of experiments were designed to determine whether the combinatorial splint could be similarly utilized to facilitate a pooled ligation reaction of all 20 trinucleotide codons or discrete subsets thereof, with no context dependence (i.e., independent of base composition of the hexamer to be inserted in the ligation reaction). Two sets of site-saturation mutagenesis primers are shown below, representing all 20 codon insertions for two distinct targets. The inserted hexamers are indicated in bold, and contain a variable trinucleotide which codes for all 20 possible amino acids and a fixed trinucleotide which codes for a specific amino acid present in the wild type sequence. The three unique oligos needed to generate each set of 20 primers are also indicated below as the L-ODN, R-ODN, and degenerate splint S-ODN, where N=dA, dC, dG, or T.

Primer Set 1.

| | | |
|---|---|---|
| Ala-Phe-Primer | 5′- CATAAGGATGGCCCACCAATTTC GCGTTC ATCTGCCGATCAGGG | (SEQ ID NO: 19) |
| Arg-Phe-Primer | 5′- CATAAGGATGGCCCACCAATTTC CGCTTC ATCTGCCGATCAGGG | (SEQ ID NO: 20) |
| Asn-Phe-Primer | 5′- CATAAGGATGGCCCACCAATTTC AACTTC ATCTGCCGATCAGGG | (SEQ ID NO: 21) |
| Asp-Phe-Primer | 5′- CATAAGGATGGCCCACCAATTTC GATTTC ATCTGCCGATCAGGG | (SEQ ID NO: 22) |
| Cys-Phe-Primer | 5′- CATAAGGATGGCCCACCAATTTC TGCTTC ATCTGCCGATCAGGG | (SEQ ID NO: 23) |
| Gln-Phe-Primer | 5′- CATAAGGATGGCCCACCAATTTC CAGTTC ATCTGCCGATCAGGG | (SEQ ID NO: 24) |
| Glu-Phe-Primer | 5′- CATAAGGATGGCCCACCAATTTC GAATTC ATCTGCCGATCAGGG | (SEQ ID NO: 25) |
| Gly-Phe-Primer | 5′- CATAAGGATGGCCCACCAATTTC GGCTTC ATCTGCCGATCAGGG | (SEQ ID NO: 26) |
| His-Phe-Primer | 5′- CATAAGGATGGCCCACCAATTTC CATTTC ATCTGCCGATCAGGG | (SEQ ID NO: 27) |
| Ile-Phe-Primer | 5′- CATAAGGATGGCCCACCAATTTC ATTTTC ATCTGCCGATCAGGG | (SEQ ID NO: 28) |
| Leu-Phe-Primer | 5′- CATAAGGATGGCCCACCAATTTC CTGTTC ATCTGCCGATCAGGG | (SEQ ID NO: 29) |
| Lys-Phe-Primer | 5′- CATAAGGATGGCCCACCAATTTC AAATTC ATCTGCCGATCAGGG | (SEQ ID NO: 30) |
| Met-Phe-Primer | 5′- CATAAGGATGGCCCACCAATTTC ATGTTC ATCTGCCGATCAGGG | (SEQ ID NO: 31) |
| Phe-Phe-Primer | 5′- CATAAGGATGGCCCACCAATTTC TTCTTC ATCTGCCGATCAGGG | (SEQ ID NO: 32) |
| Pro-Phe-Primer | 5′- CATAAGGATGGCCCACCAATTTC CCGTTC ATCTGCCGATCAGGG | (SEQ ID NO: 33) |
| Ser-Phe-Primer | 5′- CATAAGGATGGCCCACCAATTTC AGCTTC ATCTGCCGATCAGGG | (SEQ ID NO: 34) |
| Thr-Phe-Primer | 5′- CATAAGGATGGCCCACCAATTTC ACCTTC ATCTGCCGATCAGGG | (SEQ ID NO: 35) |
| Trp-Phe-Primer | 5′- CATAAGGATGGCCCACCAATTTC TGGTTC ATCTGCCGATCAGGG | (SEQ ID NO: 36) |
| Tyr-Phe-Primer | 5′- CATAAGGATGGCCCACCAATTTC TATTTC ATCTGCCGATCAGGG | (SEQ ID NO: 37) |
| Val-Phe-Primer | 5′- CATAAGGATGGCCCACCAATTTC GTGTTC ATCTGCCGATCAGGG | (SEQ ID NO: 38) |
| L-ODN | 5′- CATAAGGATGGCCCACCAATTTC | (SEQ ID NO: 39) |
| R-ODN | 5′- ATCTGCCGATCAGGG | (SEQ ID NO: 40) |
| S-ODN | 5′- ATCGGCAGATGAANNNGAAATTGGTG | (SEQ ID NO: 41) |

Primer Set 2.

| | | |
|---|---|---|
| Ala-Lys-Primer | 5′- CGATGGGAACAATCGTCC GCGAAA ACCTGGCGAATTAG | (SEQ ID NO: 42) |
| Arg-Lys-Primer | 5′- CGATGGGAACAATCGTCC CGCAAA ACCTGGCGAATTAG | (SEQ ID NO: 43) |
| Asn-Lys-Primer | 5′- CGATGGGAACAATCGTCC AACAAA ACCTGGCGAATTAG | (SEQ ID NO: 44) |
| Asp-Lys-Primer | 5′- CGATGGGAACAATCGTCC GATAAA ACCTGGCGAATTAG | (SEQ ID NO: 45) |
| Cys-Lys-Primer | 5′- CGATGGGAACAATCGTCC TGCAAA ACCTGGCGAATTAG | (SEQ ID NO: 46) |
| Gln-Lys-Primer | 5′- CGATGGGAACAATCGTCC CAGAAA ACCTGGCGAATTAG | (SEQ ID NO: 47) |
| Glu-Lys-Primer | 5′- CGATGGGAACAATCGTCC GAAAAA ACCTGGCGAATTAG | (SEQ ID NO: 48) |
| Gly-Lys-Primer | 5′- CGATGGGAACAATCGTCC GGCAAA ACCTGGCGAATTAG | (SEQ ID NO: 49) |
| His-Lys-Primer | 5′- CGATGGGAACAATCGTCC CATAAA ACCTGGCGAATTAG | (SEQ ID NO: 50) |
| Ile-Lys-Primer | 5′- CGATGGGAACAATCGTCC ATTAAA ACCTGGCGAATTAG | (SEQ ID NO: 51) |
| Leu-Lys-Primer | 5′- CGATGGGAACAATCGTCC CTGAAA ACCTGGCGAATTAG | (SEQ ID NO: 52) |
| Lys-Lys-Primer | 5′- CGATGGGAACAATCGTCC AAAAAA ACCTGGCGAATTAG | (SEQ ID NO: 53) |
| Met-Lys-Primer | 5′- CGATGGGAACAATCGTCC ATGAAA ACCTGGCGAATTAG | (SEQ ID NO: 54) |
| Phe-Lys-Primer | 5′- CGATGGGAACAATCGTCC TTCAAA ACCTGGCGAATTAG | (SEQ ID NO: 55) |
| Pro-Lys-Primer | 5′- CGATGGGAACAATCGTCC CCGAAA ACCTGGCGAATTAG | (SEQ ID NO: 56) |
| Ser-Lys-Primer | 5′- CGATGGGAACAATCGTCC AGCAAA ACCTGGCGAATTAG | (SEQ ID NO: 57) |
| Thr-Lys-Primer | 5′- CGATGGGAACAATCGTCC ACCAAA ACCTGGCGAATTAG | (SEQ ID NO: 58) |
| Trp-Lys-Primer | 5′- CGATGGGAACAATCGTCC TGGAAA ACCTGGCGAATTAG | (SEQ ID NO: 59) |
| Tyr-Lys-Primer | 5′- CGATGGGAACAATCGTCC TATAAA ACCTGGCGAATTAG | (SEQ ID NO: 60) |
| Val-Lys-Primer | 5′- CGATGGGAACAATCGTCC GTGAAA ACCTGGCGAATTAG | (SEQ ID NO: 61) |
| L-ODN | 5′- CGATGGGAACAATCGTCC | (SEQ ID NO: 62) |
| R-ODN | 5′- ACCTGGCGAATTAG | (SEQ ID NO: 63) |
| S-ODN | 5′- TCGCCAGGTTTTNNNGGACGATTG | (SEQ ID NO: 64) |

Each full-length oligonucleotide was synthesized individually by reacting the L-ODN, desired hexamer, R-ODN, and degenerate splint (i.e., the family of S-ODNs having degenerate nucleotide substitution at the locations indicated with “N“s in the above Tables) in an analogous fashion as described above for the perfectly matched template-driven reaction. Each reaction proceeded to similar extent of completion utilizing the degenerate splint, as was observed earlier with the perfectly matched template-driven reaction. Seven different pooled ligation reactions were also run, to obtain primer sets comprised of primers 1-5, 6-10, 11-15, 16-20, 1-10, 11-20, 1-20, respectively, from the two complete primer sets shown above. The products of pooled ligations (utilizing degenerate splint) were compared to the analogous statistical mix of each of the full-length primers, obtained via individual ligation reaction.

HPLC purification and analysis of the resulting chromatographic profiles, suggested that the products obtained from pooled ligation reactions were consistent with those expected for that particular set of desired ligation products, based on comparison of retention times of the products obtained from individual ligation reactions.

### Example 4

Single Ligation Reaction Synthesis of Codon-varied Oligonucleotide Pools Using a 3/3 Combinatorial Splint

The next set of experiments were designed to determine whether a combinatorial splint could similarly facilitate an individual ligation reaction, as well as a pooled ligation reaction, utilizing a trimer insertion strategy. A subset representing 5 of the 20 codon insertions from Primer Set 1 was redesigned and assessed to determine the efficiency and feasibility of this approach.

Primer Set 1a.

| | | |
|---|---|---|
| Leu-Phe Primer | 5′-CATAAGGATGGCCCACCAATTTC CTG TTCATCTGCCGATCAGGG | (SEQ ID NO: 65) |
| Lys-Phe Primer | 5′-CATAAGGATGGCCCACCAATTTC AAA TTCATCTGCCGATCAGGG | (SEQ ID NO: 66) |
| Met-Phe Primer | 5′-CATAAGGATGGCCCACCAATTTC ATG TTCATCTGCCGATCAGGG | (SEQ ID NO: 67) |
| Phe-Phe Primer | 5′-CATAAGGATGGCCCACCAATTTC TTC TTCATCTGCCGATCAGGG | (SEQ ID NO: 68) |
| Pro-Phe Primer | 5′-CATAAGGATGGCCCACCAATTTC CCG TTCATCTGCCGATCAGGG | (SEQ ID NO: 69) |
| L-ODN | 5′-CATAAGGATGGCCCACCAATTTC | (SEQ ID NO: 70) |
| R-ODN | 5′-TTCATCTGCCGATCAGGG | (SEQ ID NO: 71) |
| S-ODN | 5′-ATCGGCAGATGAANNNGAAATTGGTG | (SEQ ID NO: 72) |

The oligonucleotides used for the set of reactions indicated above were synthesized in an analogous fashion to those utilized in the original Primer Set 1 with the exception that phosphorylated trimers were obtained via chemical phosphorylation on automated DNA synthesizer (utilizing Chemical Phosphorylation Reagent and Glen Research's recommended standard protocol).

Each full-length oligonucleotide was synthesized individually by reacting the L-ODN, desired phosphorylated trimer, R-ODN, and degenerate splint in an analogous fashion as described above for perfectly matched template-driven reaction. Each trimer insertion reaction proceeded nearly to the extent of completion as those obtained utilizing the 3/6 combinatorial splint strategy to facilitate hexamer insertion. Each of the five individual full-length primers were synthesized, as well as the primer set comprised of primers 11-15 from Primer Set 1 shown above. The individual product from each of the five trimer insertion reactions and the products of pooled ligation of primers 11-15 were similarly analyzed via HPLC.

HPLC purification and analysis of the resulting chromatographic profiles, suggested that the products obtained from pooled ligation reactions were consistent with those expected for the desired ligation products (and had a similar chromatographic profile to the analogous hexamer insertion reaction corresponding to the primers 11-15 shown above), based on comparison of retention times of the products obtained from individual ligation reactions.

### Example 5

Sequence Analysis of DNA From Site-directed Mutagenesis With Codon-varied Primer Pools Made With 3/6 Combinatorial Splint

All mixed primer sets (from both pooled ligation and statistical mixes) were quantitated, diluted to 2 pmol/μL, and utilized in QuikChange multisite-directed mutagenesis (essentially the procedure described in the Stratagene (La Jolla, Calif., USA, @stratagene.com) instruction manual). The amplified products obtained from QuikChange were sequenced employing standard sequencing methods, and the results indicated that the primer sets obtained via pooled ligation afforded products with diversity similar to those generated individually and subsequently batched following quantitation (the so-called statistical mixes).

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method for preparing a polynucleotide, comprising
a) combining a left oligonucleotide (L-ODN), an intermediate oligonucleotide (I-ODN), a right oligonucleotide (R-ODN) and a splint oligonucleotide (S-ODN) to form a mixture, where
i. a 3′-most region of the L-ODN is functionally complementary to a 3′-most region of the S-ODN;
ii. a 5′-most region of the R-ODN is functionally complementary to a 5′-most region of the S-ODN;
iii. the L-ODN and the R-ODN anneal to the S-ODN to provide a gap between the 3′ end of the L-ODN and the 5′ end of the R-ODN;
iv. the I-ODN anneals to a sequence of nucleotides termed the variable region of the S-ODN and thereby fills the gap; and
b) ligating the I-ODN to both the L-ODN and the R-ODN to form a polynucleotide.

2. A method for preparing a plurality of polynucleotides, comprising
a) combining a left oligonucleotide (L-ODN), a plurality of intermediate oligonucleotides (I-ODN), a right oligonucleotide (R-ODN) and a splint oligonucleotide (S-ODN) to form a mixture, where
i. a 3′-most region of the L-ODN is functionally complementary to a 3′-most region of the S-ODN;
ii. a 5′-most region of the R-ODN is functionally complementary to a 5′-most region of the S-ODN;
iii. the L-ODN and the R-ODN anneal to the S-ODN to provide a gap between the 3′ end of the L-ODN and the 5′ end of the R-ODN;
iv. each I-ODN anneals to a sequence of nucleotides termed the variable region of the S-ODN and thereby fills the gap;
v. members of the plurality of I-ODNs have the same number of nucleotides but differ in their nucleotide sequences; and
b) ligating members of the plurality of I-ODNs to both the L-ODN and the R-ODN to form a plurality of polynucleotides.

3. A method for preparing a plurality of polynucleotides, comprising
a) combining a left oligonucleotide (L-ODN), a plurality of intermediate oligonucleotides (I-ODNs), a right oligonucleotide (R-ODN) and a plurality of splint oligonucleotide (S-ODNs) to form a mixture, where
i. a 3′-most region of the L-ODN is functionally complementary to a 3′-most region of the S-ODN;
ii. a 5′-most region of the R-ODN is functionally complementary to a 5′-most region of the S-ODN;
iii. the L-ODN and the R-ODN anneal to the S-ODN to provide a gap between the 3′ end of the L-ODN and the 5′ end of the R-ODN;
iv. each I-ODN anneals to a sequence of nucleotides termed the variable region of the S-ODN and thereby fills the gap;
v. members of the plurality of I-ODNs have the same number of nucleotides but differ in their nucleotide sequences;
vi. members of the plurality of S-ODNs have the same number of nucleotides but differ in their nucleotide sequences within the variable region of the S-ODN; and
b) ligating members of the plurality of I-ODNs to both the L-ODN and the R-ODN to form a plurality of polynucleotides.

4. The method of claim 1 wherein the L-ODN has 10-1,000 nucleotides.

5. The method of claim 4 wherein the L-ODN has 10-100 nucleotides.

6. The method of claim 1 wherein the L-ODN comprises a nucleotide sequence that is also present in a naturally occurring polynucleotide.

7. The method of claim 1 wherein the L-ODN is synthetically produced.

8. The method of claim 1 wherein the L-ODN is the only oligonucleotide in the mixture that has a 3′-most region that is functionally complementary to the 3′-most region of the S-ODN.

9. The method of claim 1 wherein the 3′-most region of the L-ODN is exactly complementary to the 3′-most region of the S-ODN.

10. The method of claim 1 wherein the 3′-most region of the L-ODN is exactly complementary to the 3′-most region of the S-ODN with the exception of one and only one mismatched base pair.

11. The method of claim 1 wherein the 3′-most region of the L-ODN that is complementary to the 3′-most region of the S-ODN has a length of 5-15 nucleotides.

12. The method of claim 1 wherein each nucleotide of the L-ODN is selected from A, G, C and T.

13. The method of claim 1 wherein the R-ODN has 10-1,000 nucleotides.

14. The method of claim 13 wherein the R-ODN has 10-100 nucleotides.

15. The method of claim 1 wherein the R-ODN comprises a nucleotide sequence that is also present in a naturally occurring polynucleotide.

16. The method of claim 1 wherein the R-ODN is synthetically produced.

17. The method of claim 1 wherein the R-ODN is the only oligonucleotide in the mixture that has a 5′-most region that is functionally complementary to the 5′-most region of the S-ODN.

18. The method of claim 1 wherein the 5′-most region of the R-ODN is exactly complementary to the 5′-most region of the S-ODN.

19. The method of claim 1 wherein the 5′-most region of the R-ODN is exactly complementary to the 5′-most region of the S-ODN with the exception of one and only one mismatched base pair.

20. The method of claim 1 wherein the 5′-most region of the R-ODN that is complementary to the 5′-most region of the S-ODN has a length of 5-15 nucleotides.

21. The method of claim 1 wherein each nucleotide of the R-ODN is selected from A, G, C and T.

22. The method of claim 1 wherein the L-ODN and the R-ODN anneal to the S-ODN to provide a gap between the 3′ end of the L-ODN and the 5′ end of the R-ODN, where the gap is across from the variable region of the S-ODN, so that the gap, the variable region of the S-ODN, and the I-ODN have the same number of nucleotides x, wherein x is an integer selected from 1-30 nucleotides.

23. The method of claim 22 wherein x is 2-20.

24. The method of claim 22 wherein x is 2-12.

25. The method of claim 22 wherein x is 3-9.

26. The method of claim 1 wherein the S-ODN has 10 to 100 nucleotides.

27. The method of claim 26 wherein the S-ODN has 15-50 nucleotides.

28. The method of claim 26 wherein the S-ODN has 18-30 nucleotides.

29. The method of claim 26 wherein the S-ODN is synthetically produced.

30. The method of claim 1 wherein ligating the I-ODN to both the L-ODN and the R-ODN to form a polynucleotide is accomplished via a ligase.

31. The method of claim 1 wherein ligating the I-ODN to both the L-ODN and the R-ODN to form a polynucleotide is accomplished without a ligase.

32. The method of claim 1 wherein the polynucleotide formed by the ligation step has 20-2,000 nucleotides.

33. The method of claim 32 wherein the polynucleotide formed by the ligation step has 30-300 nucleotides.

34. The method of claim 32 wherein the polynucleotide formed by the ligation step has 20-100 nucleotides.

35. The method of claim 2 wherein, when the mixture comprises a plurality of intermediate oligonucleotides (I-ODNs), each member of the plurality of I-ODNs has a contiguous sequence of nucleotides termed the codon region, and a contiguous sequence of nucleotides termed the fixed region, wherein each member of the plurality has the same nucleotide sequence within their fixed region, but has a different nucleotide sequence within their codon region.

36. The method of claim 35 wherein the codon region is three nucleotides in length.

37. The method of claim 35 wherein the codon region is six nucleotides in length.

38. The method of claim 35 wherein the fixed region is three nucleotides in length.

39. The method of claim 35 wherein the fixed region is six nucleotides in length.

40. The method of claim 35 wherein each I-ODN comprises two fixed regions.

41. The method of claim 35 wherein each I-ODN comprises two codon regions.

42. The method of claim 35 wherein each I-ODN has one and only one codon region, and the codon regions of the plurality of I-ODNs code for different amino acids.

43. The method of claim 35 wherein the plurality of I-ODNs has less than 21 members.

44. The method of claim 35 wherein the plurality of I-ODNs has more than 5 members.

45. The method of claim 1 wherein a number z base pairs are formed when the I-ODN anneals to the variable region of the S-ODN, and less than or equal to 0.5z of these base pairs are standard Watson-Crick base pairs.

46. The method of claim 3 wherein, when the mixture comprises a plurality of splint oligonucleotides (S-ODNs), the S-ODN plurality comprises four members, and these four members have degenerate nucleotide substitution with the variable region and at a distance of y nucleotides from the 3′ end of the variable region, i.e., these four oligonucleotides have A, G, C, and T nucleotides, respectively, at a distance of y nucleotides from the 3′ end of the variable region.

47. The method of claim 46 wherein the S-ODN plurality comprises sixteen members, and these sixteen members have degenerate nucleotide substitution within the variable region, at a distance y and at a distance y+1 nucleotides from the 3′ end of the variable region, i.e., these sixteen members comprise:
a) 4 members in a first set, where members of the first set have the A/A, A/T, A/G, and A/C nucleotides at a distance of y/y+1 nucleotides from the 3′ end of the variable region;
b) 4 members in a second set, where members of the second set have the G/A, G/T, G/G, and G/C nucleotides at a distance of y/y+1 nucleotides from the 3′ end of the variable region;
c) 4 members in a third set, where members of the third set have the C/A, C/T, C/G, and C/C nucleotides at a distance of y/y+1 nucleotides from the 3′ end of the variable region; and
d) 4 members in a fourth set, where members of the fourth set have the T/A, T/T, T/G, and T/C nucleotides at a distance of y/y+1 nucleotides from the 3′ end of the variable region.

48. The method of claim 46 wherein the S-ODN plurality comprises sixty four members, and these sixty four members have degenerate nucleotide substitution within the variable region at a distance y, and at a distance y+1, and at a distance y+2 nucleotides from the 3′ end of the variable region.

49. The method of claim 46 wherein members of the plurality of S-ODNs have degenerate nucleotide substitution at nucleotide locations that base pair with nucleotides in the codon regions of the I-ODNs.

50. The method of claim 46 wherein members of the plurality of S-ODNs have degenerate nucleotide substitution at 2 out of 3 nucleotide locations that base pair with nucleotides in the 3 nucleotide-containing codon regions of the I-ODNs.

51. The method of claim 2 wherein, when a plurality of polynucleotides are formed, the plurality has 2-20 members.

52. The method of claim 2 wherein, when a plurality of polynucleotides are formed, the plurality has 5-20 members.

53. The method of claim 2 wherein, when a plurality of polynucleotides are formed, the plurality has less than 64 members.

54. The method of claim 2 wherein, when a plurality of polynucleotides are formed, the plurality has less than 60 members.

55. The method of claim 2 wherein, when a plurality of polynucleotides are formed, the plurality has less than 50 members.

56. The method of claim 2 wherein members of the plurality of polynucleotides each have the same number of nucleotides, but differ from one another in that three nucleotides located a distance m, m+1 and m+2 nucleotides from the 3′ end of each polynucleotide together encode for different amino acids.

57. A composition comprising a left oligonucleotide (L-ODN), an intermediate oligonucleotide (I-ODN), a right oligonucleotide (R-ODN) and a splint oligonucleotide (S-ODN), wherein
i. a 3′-most region of the L-ODN is functionally complementary to a 3′-most region of the S-ODN;
ii. a 5′-most region of the R-ODN is functionally complementary to a 5′-most region of the S-ODN;
iii. when the 3′-most region of the L-ODN anneals to the 3′-most region of the S-ODN, and the 5′-most region of the R-ODN anneals to the 5′-most region of the S-ODN, a gap is formed between the 3′ end of the L-ODN and the 5′ end of the R-ODN; and
iv. the I-ODN has a nucleotide sequence that allows it to anneal to a sequence of nucleotides termed the variable region of the S-ODN, where the gap is located across from the variable region.

58. A composition comprising a left oligonucleotide (L-ODN), a plurality of intermediate oligonucleotides (I-ODN), a right oligonucleotide (R-ODN) and a splint oligonucleotide (S-ODN), wherein
i. a 3′-most region of the L-ODN is functionally complementary to a 3′-most region of the S-ODN;
ii. a 5′-most region of the R-ODN is functionally complementary to a 5′-most region of the S-ODN;
iii. when the 3′-most region of the L-ODN anneals to the 3′-most region of the S-ODN, and the 5′-most region of the R-ODN anneals to the 5′-most region of the S-ODN, a gap is formed between the 3′ end of the L-ODN and the 5′ end of the R-ODN; and
iv. each I-ODN has a nucleotide sequence that allows it to anneal to a sequence of nucleotides termed the variable region of the S-ODN, where the gap is located across from the variable region; and
v. members of the plurality of I-ODNs have the same number of nucleotides but differ in their nucleotide sequences.

59. A composition comprising a left oligonucleotide (L-ODN), a plurality of intermediate oligonucleotides (I-ODNs), a right oligonucleotide (R-ODN) and a plurality of splint oligonucleotide (S-ODNs), wherein
i. a 3′-most region of the L-ODN is functionally complementary to a 3′-most region of the S-ODNs;
ii. a 5′-most region of the R-ODN is functionally complementary to a 5′-most region of the S-ODNs;
iii. when the 3′-most region of the L-ODN anneals to the 3′-most region of the S-ODNs, and the 5′-most region of the R-ODN anneals to the 5′-most region of the S-ODNs, a gap is formed between the 3′ end of the L-ODN and the 5′ end of the R-ODN; and
iv. each I-ODN has a nucleotide sequence that allows it to anneal to a sequence of nucleotides termed the variable region of the S-ODNs, where the gap is located across from the variable region;
v. members of the plurality of I-ODNs have the same number of nucleotides but differ in their nucleotide sequences; and
vi. members of the plurality of S-ODNs have the same number of nucleotides but differ in their nucleotide sequences within the variable region of the S-ODN.

60. The composition of claim 57 wherein the L-ODN has 10-1,000 nucleotides.

61. The composition of claim 60 wherein the L-ODN has 10-100 nucleotides.

62. The composition of claim 57 wherein the L-ODN comprises a nucleotide sequence that is also present in a naturally occurring polynucleotide.

63. The composition of claim 57 wherein the L-ODN is synthetically produced.

64. The composition of claim 57 wherein the L-ODN is the only oligonucleotide in the mixture that has a 3′-most region that is functionally complementary to the 3′-most region of the S-ODN.

65. The composition of claim 57 wherein the 3′-most region of the L-ODN is exactly complementary to the 3′-most region of the S-ODN.

66. The composition of claim 57 wherein the 3′-most region of the L-ODN is exactly complementary to the 31-most region of the S-ODN(s) with the exception of one and only one mismatched base pair.

67. The composition of claim 57 wherein the 3′-most region of the L-ODN that is complementary to the 3′-most region of the S-ODN has a length of 5-15 nucleotides.

68. The composition of claim 57 wherein each nucleotide of the L-ODN is selected from A, G, C and T.

69. The composition of claim 57 wherein the R-ODN has 10-1000 nucleotides.

70. The composition of claim 69 wherein the R-ODN has 10-100 nucleotides.

71. The composition of claim 57 wherein the R-ODN comprises a nucleotide sequence that is also present in a naturally occurring polynucleotide.

72. The composition of claim 57 wherein the R-ODN is synthetically produced.

73. The composition of claim 57 wherein the R-ODN is the only oligonucleotide in the mixture that has a 5′-most region that is functionally complementary to the 5′-most region of the S-ODN.

74. The composition of claim 57 wherein the 5′-most region of the R-ODN is exactly complementary to the 5′-most region of the S-ODN.

75. The composition of claim 57 wherein the 5′-most region of the R-ODN is exactly complementary to the 5′-most region of the S-ODN with the exception of one and only one mismatched base pair.

76. The composition of claim 57 wherein the 5′-most region of the R-ODN that is complementary to the 5′-most region of the S-ODN has a length of 5-15 nucleotides.

77. The composition of claim 57 wherein each nucleotide of the R-ODN is selected from A, G, C and T.

78. The composition of claim 57 wherein the L-ODN and the R-ODN anneal to the S-ODN to provide a gap between the 3′ end of the L-ODN and the 5′ end of the R-ODN, where the gap is across from the variable region of the S-ODN, so that the gap, the variable region of the S-ODN, and the I-ODN have the same number of nucleotides x, wherein x is an integer selected from 1-30 nucleotides.

79. The composition of claim 78 wherein x is 2-20.

80. The composition of claim 79 wherein x is 2-12.

81. The composition of claim 79 wherein x is 3-9.

82. The composition of claim 57 wherein the S-ODN has 10 to 100 nucleotides.

83. The composition of claim 82 wherein the S-ODN has 15-50 nucleotides.

84. The composition of claim 82 wherein the S-ODN has 18-30 nucleotides.

85. The composition of claim 57 wherein the S-ODN is synthetically produced.

86. The composition of claims 57 further comprising a ligase.

87. The composition of claim 57 further comprising one or more chemical reagents that can achieve chemical ligation of the I-ODN to both the L-ODN and the S-ODN.

88. The composition of claim 57 further comprising a polynucleotide that comprises the nucleotide sequences of the R-ODN, the I-ODN and the L-ODN, where the polynucleotide has 20-2,000 nucleotides.

89. The composition of claim 88 wherein the polynucleotide has 30-300 nucleotides.

90. The composition of claim 88 wherein the polynucleotide has 20-100 nucleotides.

91. The composition of claim 58 wherein, when the mixture comprises a plurality of intermediate oligonucleotides (I-ODNs), each member of the plurality of I-ODNs has a contiguous sequence of nucleotides termed the codon region, and a contiguous sequence of nucleotides termed the fixed region, wherein each member of the plurality has the same nucleotide sequence within their fixed region, but has a different nucleotide sequence within their codon region.

92. The composition of claim 91 wherein the codon region is three nucleotides in length.

93. The composition of claim 91 wherein the codon region is six nucleotides in length.

94. The composition of claim 91 wherein the fixed region is three nucleotides in length.

95. The composition of claim 91 wherein the fixed region is six nucleotides in length.

96. The composition of claim 91 wherein each I-ODN comprises two fixed regions.

97. The composition of claim 91 wherein each I-ODN comprises two codon regions.

98. The composition of claim 91 wherein each I-ODN has one and only one codon region, and the codon regions of the plurality of I-ODNs code for different amino acids.

99. The composition of claim 91 wherein the plurality of I-ODNs has less than 21 members.

100. The composition of claim 91 wherein the plurality of I-ODNs has more than 5 members.

101. The composition of claim 57 wherein a number z base pairs are formed when the I-ODN anneals to the variable region of the S-ODN, and less than or equal to 0.5z of these base pairs are standard Watson-Crick base pairs.

102. The composition of claim 59 wherein, when the mixture comprises a plurality of splint oligonucleotides (S-ODNs), the S-ODN plurality comprises four members, and these four members have degenerate nucleotide substitution with the variable region and at a distance of y nucleotides from the 3′ end of the variable region, i.e., these four oligonucleotides have A, G, C, and T nucleotides, respectively, at a distance of y nucleotides from the 3′ end of the variable region.

103. The composition of claim 102 wherein the S-ODN plurality comprises sixteen members, and these sixteen members have degenerate nucleotide substitution within the variable region, at a distance y and at a distance y+1 nucleotides from the 3′ end of the variable region, i.e., these sixteen members comprise:
a) 4 members in a first set, where members of the first set have the A/A, A/T, A/G, and A/C nucleotides at a distance of y/y+1 nucleotides from the 3′ end of the variable region;
b) 4 members in a second set, where members of the second set have the G/A, G/T, G/G, and G/C nucleotides at a distance of y/y+1 nucleotides from the 3′ end of the variable region;
c) 4 members in a third set, where members of the third set have the C/A, C/T, C/G, and C/C nucleotides at a distance of y/y+1 nucleotides from the 3′ end of the variable region; and
d) 4 members in a fourth set, where members of the fourth set have the T/A, T/T, T/G, and T/C nucleotides at a distance of y/y+1 nucleotides from the 3′ end of the variable region.

104. The composition of claim 102 wherein the S-ODN plurality comprises sixty four members, and these sixty four members have degenerate nucleotide substitution within the variable region at a distance y, and at a distance y+1, and at a distance y+2 nucleotides from the 3′ end of the variable region.

105. The composition of claim 102 wherein members of the plurality of S-ODNs have degenerate nucleotide substitution at nucleotide locations that base pair with nucleotides in the codon regions of the I-ODNs.

106. The composition of claim 102 wherein members of the plurality of S-ODNs have degenerate nucleotide substitution at 2 out of 3 nucleotide locations that base pair with nucleotides in the 3 nucleotide-containing codon regions of the I-ODNs.

107. A composition comprising a plurality of oligonucleotides, wherein each member of the plurality comprises a nucleotide sequence extending from a 3′ end to a 5′ end of the oligonucleotide, and
i. the nucleotide sequence of each oliognucleotide in the plurality consists of a R-ODN-derived sequence including the 3′-most nucleotide of the oligonucleotide, an I-ODN-derived sequence located between the R-ODN-derived sequence and the L-ODN-derived sequence, and a L-ODN-derived sequence including the 5′-most nucleotide of the oligonucleotide;
ii. the R-ODN-derived sequences of each member of the plurality are identical;
iii. the L-ODN-derived sequences of each member of the plurality are identical; and
iv. the I-ODN-derived sequences of each member of the plurality are different.


SEQUENCE LISTING (72 sequences)

SEQ ID NO: 1 - DNA, 23 bp, Artificial Sequence
  Oligonucleotide primer
cataaggatggcccaccaatttc

SEQ ID NO: 2 - DNA, 15 bp, Artificial Sequence
  Oligonucleotide primer
atctgccgatcaggg

SEQ ID NO: 3 - DNA, 26 bp, Artificial Sequence
  Splint molecule - 10 bases at the 3′end fo the L-ODN, six bases of the hexamer and 10 bases at the 5′-end of R-ODN.
atcggcagatgaacgcgaaattggtg

SEQ ID NO: 4 - DNA, 23 bp, Artificial Sequence
  Oligonucleotide primer
cataaggatggcccaccaatttc

SEQ ID NO: 5 - DNA, 15 bp, Artificial Sequence
  Oligonucleotide primer
atctgccgatcaggg

SEQ ID NO: 6 - DNA, 26 bp, Artificial Sequence
  Splint molecule - 10 bases at the 3′end fo the L-ODN, six bases of the hexamer and 10 bases at the 5′-end of R-ODN.
atcggcagatgaagcggaaattggtg

SEQ ID NO: 7 - DNA, 23 bp, Artificial Sequence
  Oligonucleotide primer
cataaggatggcccaccaatttc

SEQ ID NO: 8 - DNA, 15 bp, Artificial Sequence
  Oligonucleotide primer
atctgccgatcaggg

SEQ ID NO: 9 - DNA, 26 bp, Artificial Sequence
  Splint molecule - 10 bases at the 3′end fo the L-ODN, six bases of the hexamer and 10 bases at the 5′-end of R-ODN.
atcggcagatgaagttgaaattggtg

SEQ ID NO: 10 - DNA, 23 bp, Artificial Sequence
  Oligonucleotide primer
cataaggatggcccaccaatttc

SEQ ID NO: 11 - DNA, 15 bp, Artificial Sequence
  Oligonucleotide primer
atctgccgatcaggg

SEQ ID NO: 12 - DNA, 26 bp, Artificial Sequence
  Splint molecule - 10 bases at the 3′end fo the L-ODN, six bases of the hexamer and 10 bases at the 5′-end of R-ODN.
atcggcagatgaaatcgaaattggtg

SEQ ID NO: 13 - DNA, 23 bp, Artificial Sequence
  Oligonucleotide primer
cataaggatggcccaccaatttc

SEQ ID NO: 14 - DNA, 15 bp, Artificial Sequence
  Oligonucleotide primer
atctgccgatcaggg

SEQ ID NO: 15 - DNA, 26 bp, Artificial Sequence
  Splint molecule - 10 bases at the 3′end fo the L-ODN, six bases of the hexamer and 10 bases at the 5′-end of R-ODN.
atcggcagatgaagcagaaattggtg

SEQ ID NO: 16 - DNA, 23 bp, Artificial Sequence
  Oligonucleotide primer
cataaggatggcccaccaatttc

SEQ ID NO: 17 - DNA, 15 bp, Artificial Sequence
  Oligonucleotide primer
atctgccgatcaggg

SEQ ID NO: 18 - DNA, 26 bp, Artificial Sequence
  Splint molecule - 10 bases at the 3′end fo the L-ODN, six bases of the hexamer and 10 bases at the 5′-end of R-ODN.
atcggcagatgaactggaaattggtg

SEQ ID NO: 19 - DNA, 44 bp, Artificial Sequence
  site-saturation mutagenesis primer
cataaggatggcccaccaatttcgcgttcatctgccgatcaggg

SEQ ID NO: 20 - DNA, 44 bp, Artificial Sequence
  site-saturation mutagenesis primer
cataaggatggcccaccaatttccgcttcatctgccgatcaggg

SEQ ID NO: 21 - DNA, 44 bp, Artificial Sequence
  site-saturation mutagenesis primer
cataaggatggcccaccaatttcaacttcatctgccgatcaggg

SEQ ID NO: 22 - DNA, 44 bp, Artificial Sequence
  site-saturation mutagenesis primer
cataaggatggcccaccaatttcgatttcatctgccgatcaggg

SEQ ID NO: 23 - DNA, 44 bp, Artificial Sequence
  site-saturation mutagenesis primer
cataaggatggcccaccaatttctgcttcatctgccgatcaggg

SEQ ID NO: 24 - DNA, 44 bp, Artificial Sequence
  site-saturation mutagenesis primer
cataaggatggcccaccaatttccagttcatctgccgatcaggg

SEQ ID NO: 25 - DNA, 44 bp, Artificial Sequence
  site-saturation mutagenesis primer
cataaggatggcccaccaatttcgaattcatctgccgatcaggg

SEQ ID NO: 26 - DNA, 44 bp, Artificial Sequence
  site-saturation mutagenesis primer
cataaggatggcccaccaatttcggcttcatctgccgatcaggg

SEQ ID NO: 27 - DNA, 44 bp, Artificial Sequence
  site-saturation mutagenesis primer
cataaggatggcccaccaatttccatttcatctgccgatcaggg

SEQ ID NO: 28 - DNA, 44 bp, Artificial Sequence
  site-saturation mutagenesis primer
cataaggatggcccaccaatttcattttcatctgccgatcaggg

SEQ ID NO: 29 - DNA, 44 bp, Artificial Sequence
  site-saturation mutagenesis primer
cataaggatggcccaccaatttcctgttcatctgccgatcaggg

SEQ ID NO: 30 - DNA, 44 bp, Artificial Sequence
  site-saturation mutagenesis primer
cataaggatggcccaccaatttcaaattcatctgccgatcaggg

SEQ ID NO: 31 - DNA, 44 bp, Artificial Sequence
  site-saturation mutagenesis primer
cataaggatggcccaccaatttcatgttcatctgccgatcaggg

SEQ ID NO: 32 - DNA, 44 bp, Artificial Sequence
  site-saturation mutagenesis primer
cataaggatggcccaccaatttcttcttcatctgccgatcaggg

SEQ ID NO: 33 - DNA, 44 bp, Artificial Sequence
  site-saturation mutagenesis primer
cataaggatggcccaccaatttcccgttcatctgccgatcaggg

SEQ ID NO: 34 - DNA, 44 bp, Artificial Sequence
  site-saturation mutagenesis primer
cataaggatggcccaccaatttcagcttcatctgccgatcaggg

SEQ ID NO: 35 - DNA, 44 bp, Artificial Sequence
  site-saturation mutagenesis primer
cataaggatggcccaccaatttcaccttcatctgccgatcaggg

SEQ ID NO: 36 - DNA, 44 bp, Artificial Sequence
  site-saturation mutagenesis primer
cataaggatggcccaccaatttctggttcatctgccgatcaggg

SEQ ID NO: 37 - DNA, 44 bp, Artificial Sequence
  site-saturation mutagenesis primer
cataaggatggcccaccaatttctatttcatctgccgatcaggg

SEQ ID NO: 38 - DNA, 44 bp, Artificial Sequence
  site-saturation mutagenesis primer
cataaggatggcccaccaatttcgtgttcatctgccgatcaggg

SEQ ID NO: 39 - DNA, 23 bp, Artificial Sequence
  site-saturation mutagenesis primer
cataaggatggcccaccaatttc

SEQ ID NO: 40 - DNA, 15 bp, Artificial Sequence
  site-saturation mutagenesis primer
atctgccgatcaggg

SEQ ID NO: 41 - DNA, 26 bp, Artificial Sequence
  site-saturation mutagenesis primer
atcggcagatgaannngaaattggtg

SEQ ID NO: 42 - DNA, 38 bp, Artificial Sequence
  site-saturation mutagenesis primer
cgatgggaacaatcgtccgcgaaaacctggcgaattag

SEQ ID NO: 43 - DNA, 38 bp, Artificial Sequence
  site-saturation mutagenesis primer
cgatgggaacaatcgtcccgcaaaacctggcgaattag

SEQ ID NO: 44 - DNA, 38 bp, Artificial Sequence
  site-saturation mutagenesis primer
cgatgggaacaatcgtccaacaaaacctggcgaattag

SEQ ID NO: 45 - DNA, 38 bp, Artificial Sequence
  site-saturation mutagenesis primer
cgatgggaacaatcgtccgataaaacctggcgaattag

SEQ ID NO: 46 - DNA, 38 bp, Artificial Sequence
  site-saturation mutagenesis primer
cgatgggaacaatcgtcctgcaaaacctggcgaattag

SEQ ID NO: 47 - DNA, 38 bp, Artificial Sequence
  site-saturation mutagenesis primer
cgatgggaacaatcgtcccagaaaacctggcgaattag

SEQ ID NO: 48 - DNA, 38 bp, Artificial Sequence
  site-saturation mutagenesis primer
cgatgggaacaatcgtccgaaaaaacctggcgaattag

SEQ ID NO: 49 - DNA, 38 bp, Artificial Sequence
  site-saturation mutagenesis primer
cgatgggaacaatcgtccggcaaaacctggcgaattag

SEQ ID NO: 50 - DNA, 38 bp, Artificial Sequence
  site-saturation mutagenesis primer
cgatgggaacaatcgtcccataaaacctggcgaattag

SEQ ID NO: 51 - DNA, 38 bp, Artificial Sequence
  site-saturation mutagenesis primer
cgatgggaacaatcgtccattaaaacctggcgaattag

SEQ ID NO: 52 - DNA, 38 bp, Artificial Sequence
  site-saturation mutagenesis primer
cgatgggaacaatcgtccctgaaaacctggcgaattag

SEQ ID NO: 53 - DNA, 38 bp, Artificial Sequence
  site-saturation mutagenesis primer
cgatgggaacaatcgtccaaaaaaacctggcgaattag

SEQ ID NO: 54 - DNA, 38 bp, Artificial Sequence
  site-saturation mutagenesis primer
cgatgggaacaatcgtccatgaaaacctggcgaattag

SEQ ID NO: 55 - DNA, 38 bp, Artificial Sequence
  site-saturation mutagenesis primer
cgatgggaacaatcgtccttcaaaacctggcgaattag

SEQ ID NO: 56 - DNA, 38 bp, Artificial Sequence
  site-saturation mutagenesis primer
cgatgggaacaatcgtccccgaaaacctggcgaattag

SEQ ID NO: 57 - DNA, 38 bp, Artificial Sequence
  site-saturation mutagenesis primer
cgatgggaacaatcgtccagcaaaacctggcgaattag

SEQ ID NO: 58 - DNA, 38 bp, Artificial Sequence
  site-saturation mutagenesis primer
cgatgggaacaatcgtccaccaaaacctggcgaattag

SEQ ID NO: 59 - DNA, 38 bp, Artificial Sequence
  site-saturation mutagenesis primer
cgatgggaacaatcgtcctggaaaacctggcgaattag

SEQ ID NO: 60 - DNA, 38 bp, Artificial Sequence
  site-saturation mutagenesis primer
cgatgggaacaatcgtcctataaaacctggcgaattag

SEQ ID NO: 61 - DNA, 38 bp, Artificial Sequence
  site-saturation mutagenesis primer
cgatgggaacaatcgtccgtgaaaacctggcgaattag

SEQ ID NO: 62 - DNA, 18 bp, Artificial Sequence
  site-saturation mutagenesis primer
cgatgggaacaatcgtcc

SEQ ID NO: 63 - DNA, 14 bp, Artificial Sequence
  site-saturation mutagenesis primer
acctggcgaattag

SEQ ID NO: 64 - DNA, 24 bp, Artificial Sequence
  site-saturation mutagenesis primer
tcgccaggttttnnnggacgattg

SEQ ID NO: 65 - DNA, 44 bp, Artificial Sequence; identical to SEQ ID NO: 29
  site-saturation mutagenesis primer

SEQ ID NO: 66 - DNA, 44 bp, Artificial Sequence; identical to SEQ ID NO: 30
  site-saturation mutagenesis primer

SEQ ID NO: 67 - DNA, 44 bp, Artificial Sequence; identical to SEQ ID NO: 31
  site-saturation mutagenesis primer

SEQ ID NO: 68 - DNA, 44 bp, Artificial Sequence; identical to SEQ ID NO: 32
  site-saturation mutagenesis primer

SEQ ID NO: 69 - DNA, 44 bp, Artificial Sequence; identical to SEQ ID NO: 33
  site-saturation mutagenesis primer

SEQ ID NO: 70 - DNA, 23 bp, Artificial Sequence
  site-saturation mutagenesis primer
cataaggatggcccaccaatttc

SEQ ID NO: 71 - DNA, 18 bp, Artificial Sequence
  site-saturation mutagenesis primer
ttcatctgccgatcaggg

SEQ ID NO: 72 - DNA, 26 bp, Artificial Sequence
  site-saturation mutagenesis primer
atcggcagatgaannngaaattggtg